(19) United States
(12) Patent Application Publication
(10) Pub. No.: US 2017/0217996 A1
(43) Pub. Date: Aug. 3, 2017

(54) Large-scale diastereoselective syntheses of cycloheptadienylsulfones and stereotetrads

(71) Applicant: Purdue Research Foundation, West Lafayette, IN (US)

(72) Inventors: Philip L. Fuchs, West Lafayette, IN (US); Mohammad Noshi, West Lafayette, IN (US)

(73) Assignee: Purdue Research Foundation, West Lafayette, IN (US)

(21) Appl. No.: 15/478,276

(22) Filed: Apr. 4, 2017

Related U.S. Application Data
(63) Continuation-in-part of application No. PCT/US2015/054375, filed on Oct. 7, 2015.
(60) Provisional application No. 62/060,771, filed on Oct. 7, 2014.

(51) Int. Cl. C07F 7/18; C07C 317/12

(52) U.S. Cl. CPC C07F 7/1848; C07C 317/12

(57) ABSTRACT

The invention relates to processes for large-scale diastereoselective syntheses of cycloheptadienylsulfone and stereotetrads, key intermediates for the preparation of Aplyronine A.

### CROSS-REFERENCE TO RELATED APPLICATIONS

This application is a continuation-in-part of International Application No. PCT/US2015/054375, filed Oct. 7, 2015, which is related to and claims the priority benefit of U.S. Provisional Patent Application Ser. No. 62/060,771, filed Oct. 7, 2014, the contents of which is hereby incorporated by reference in its entirety into this disclosure.

### FIELD OF THE INVENTION

The invention relates to processes for large-scale diastereoselective syntheses of cycloheptadienylsulfones and stereotetrads, key intermediates for the preparation of Aplyronine A.

### BACKGROUND OF THE INVENTION

Aplyronine A (ApA) is an exceptionally scarce macrolide. It has actin binding and depolymerizing properties. ApA is of potential interest as anticancer agent since actin is involved in numerous cell processes that are altered during tumorogenesis such as cytokinesis and apoptosis. Moreover, tumor invasion and metastasis are increasingly being associated with deregulation of the actin system.

ApA and ApC (a derivative of ApA that lacks a trimethylserine ester moiety) inhibit actin polymerization in vitro to the same extent, yet only ApA shows potent cytotoxicity. ApA inhibits tubulin polymerization in a unique unprecedented way. It forms a 1:1:1 heterotrimeric complex with actin and tubulin. In association with actin, ApA synergistically binds to tubulin and inhibits tubulin polymerization.

Tubulin-targeting agents have been widely used in cancer chemotherapy (e.g., Taxols and vinca alkaloids). But there are no previous descriptions of microtubule inhibitors that also bind to actin and affect microtubule assembly.

ApA inhibits spindle formation and mitosis in HeLa S3 cells at 100 pM, a much lower concentration than is needed for the disassembly of the actin cytoskeleton. This makes ApA a rare type of natural product, which binds to two different cytoplasmic proteins to exert highly potent biological activities (Kita, et al. J. Am. Chem. Soc. 2013, 135, 18089-18095). ApA increases the lifespan of mice between 201-566% in 5 different tumor models (566%: Lewis lung; 545%: P388 leukemia; 398% Ehrlich carcinoma; 255%: Colon 26 carcinoma; and 201%: B16 melanoma) (Kurodai, et al. J. Am. Chem. Soc. 1993, 115, 11020-11021).

Despite this unprecedented in vivo anticancer activity, Aplyronine A has not yet been introduced to clinical trials due to the lack of a robust and scalable methodology for its synthesis. In order to study the anti-cancer mechanism of ApA in more detail and to possibly introduce it to clinical trials, substantial quantities of aplyronine must be easily accessible via chemical synthesis. Optimization on practical synthetic scales is an essential stage in the quest for synthesis of aplyronine A analogs and bio-tools derived therefrom. Accordingly, there exists a need for a powerful methodology that enables large-scale access to key intermediates of aplyronine A and avoids expensive purification techniques.

### SUMMARY OF THE INVENTION

This invention relates to processes for large-scale diastereoselective syntheses of cycloheptadienylsulfone and stereotetrads. These compounds are key intermediates for the preparation of Aplyronine A.

In one aspect, the present invention provides a compound of formula (XI)

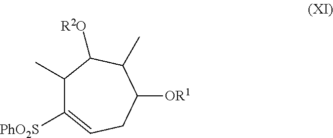

wherein R¹ and R² are independently H or a hydroxyl protecting group, wherein R¹ and R² can be same or different hydroxyl protecting group.

In one aspect, the present invention provides a process for preparing a compound of formula (I)

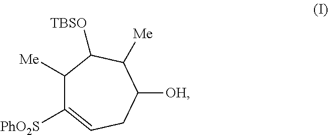

wherein the process comprising:

(a) treatment of a compound of formula (II)

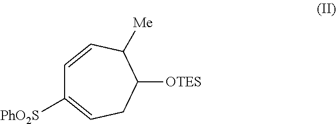

under an epoxidation reaction condition to prepare a compound of formula (III)

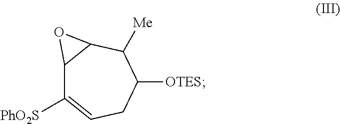

(b) treatment of said compound of formula (III) under a Lawton S_{N}2′ reaction condition with 3,5-dimethylpyrazole to prepare a compound of formula (IV)

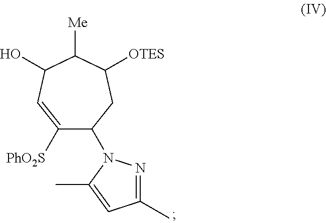

(c) Reacting said compound of formula (IV) with a Grignard reagent to prepare a compound of formula (V)

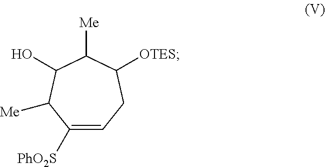

and

(d) silylation of said compound of formula (V) with TBSOTf, followed by a regioselective desilylation reaction to prepare the compound of formula (I).

In another aspect, the present invention provides a process for preparing a compound of formula (II)

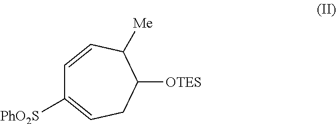

the process comprising treatment of a compound of formula (VII)

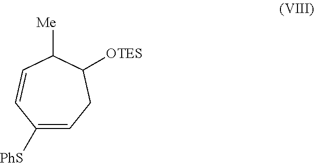

under an oxidation reaction condition.

In yet another aspect, the present invention provides a process for preparing compound (16)

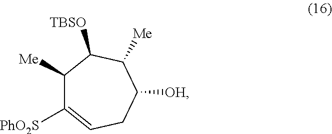

the process comprising:

(a) treatment of compound (10)

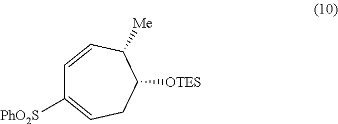

under an epoxidation reaction condition to prepare compound (13)

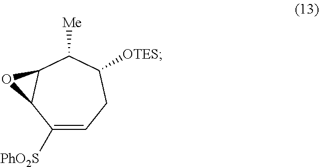

(b) treatment of said compound (13) under a Lawton S_{N}2′ reaction condition with 3,5-dimethylpyrazole to prepare compound (14)

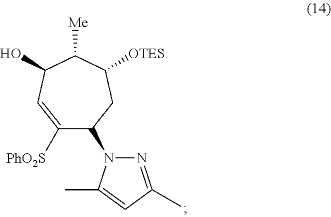

(c) Reacting said compound (14) with a Grignard reagent to prepare compound (15)

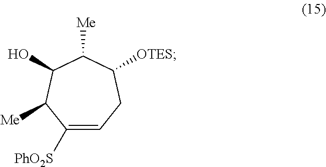

and

(d) silylation of said compound (15) with TBSOTf, followed by a regioselective desilylation reaction to prepare compound (16).

In yet another aspect, the present invention provides a process for preparing compound (2)

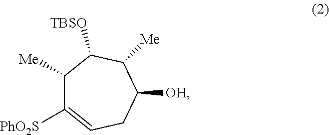

the process comprising:

(a) treatment of compound (22)

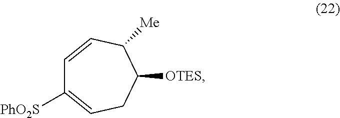

under an epoxidation reaction condition to prepare compound (25-OTES)

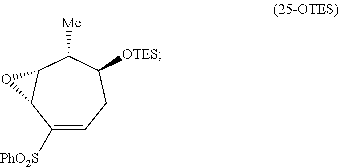

(b) treatment of said compound (25-OTES) under a Lawton S_{N}2′ reaction condition with 3,5-dimethylpyrazole to prepare compound (27)

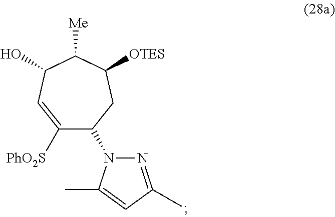

(c) Reacting said compound (27) with a Grignard reagent to prepare compound (28a)

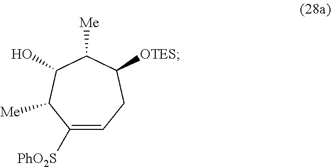

and

(d) silylation of said compound (28a) with TBSOTf, followed by a regioselective desilylation reaction to prepare compound (2).

In yet another aspect, the present invention provides a process for preparing compound (ent-2)

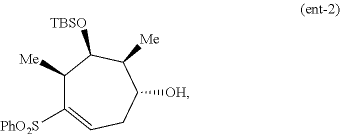

the process comprising:

(a) treatment of compound (ent-22)

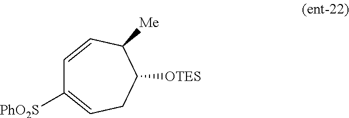

under an epoxidation reaction condition to prepare compound (ent-25-OTES)

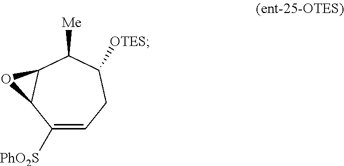

(b) treatment of said compound (ent-25-OTES) under a Lawton S_{N}2′ reaction condition with 3,5-dimethylpyrazole to prepare compound (ent-27)

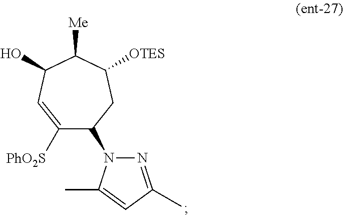

(c) Reacting said compound (ent-27) with a Grignard reagent to prepare compound (ent-28a)

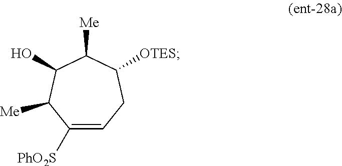

and

(d) silylation of said compound (ent-28a) with TBSOTf, followed by a regioselective desilylation reaction to prepare compound (ent-2).

Other features and advantages of the present invention will become apparent from the following detailed description examples. It should be understood, however, that the detailed description and the specific examples while indicating preferred embodiments of the invention are given by way of illustration only, since various changes and modifications within the spirit and scope of the invention will become apparent to those skilled in the art from this detailed description.

### DETAILED DESCRIPTION OF THE INVENTION

In one aspect, the present invention provides a compound of formula (XI)

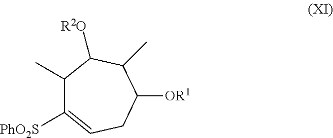

wherein R¹ and R² are independently H or a hydroxyl protecting group, wherein R¹ and R² can be same or different hydroxyl protecting group.

In one aspect, the present invention provides a compound of formula (XII)

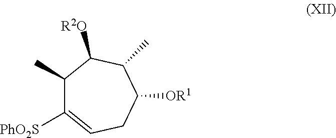

wherein R¹ and R² are independently H or a hydroxyl protecting group, wherein R¹ and R² can be same or different hydroxyl protecting group.

In one aspect, the present invention provides a compound of formula (XIII)

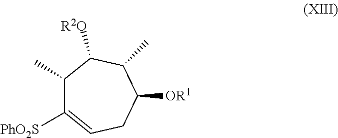

wherein R¹ and R² are independently H or a hydroxyl protecting group, wherein R¹ and R² can be same or different hydroxyl protecting group.

In one aspect, the present invention provides a compound of formula (XIV)

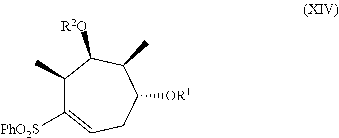

wherein R¹ and R² are independently H or a hydroxyl protecting group, wherein R¹ and R² can be same or different hydroxyl protecting group.

In any compound of formula (XI) to (XIV), wherein the hydroxyl protecting group and the hydroxyl group being protected forms a C—O ether bond, a —Si—O silyl ether bond, or —(C═O)—O acyl bond, a —SO₂—O— sulfonyl bond, a —B—O boronyl bond, a —P—O phosphate bond, or any combination thereof.

In any compound of formula (XI) to (XIV), wherein the protecting group is selected from the group consisting of trimethylsilyl (TMS), triethylsilyl (TES), tert-butyl-dimethylsilyl (TBS), triisopropylsilyl (TIPS), tert-butyl-diphenylsilyl (TBDPS), triphenylsilyl, dimethylphenylsilyl, methyldiphenylsilyl, acetyl (Ac), pivaloyl (piv), trichloroacetyl, 2,2,2-trichloroethoxycarbonyl (Troc), benzyl, p-methoxybenzyl (PMB), 3-phenylsulfonylpropionyl, benzoyl (Bz), benzyl (Bn), beta-methoxyethoxyl (MEM), dimethoxytrityl (DMT), methoxymethyl (MOM), p-methoxybenzyl (PMB), tetrahydropyranyl (THP), tetrahydrofuranyl (THF), ethoxyethyl (EE), and any combination thereof.

In one aspect, the present invention provides a composition comprising a compound of formula (XI).

The present invention provides a process for preparing a compound of formula (I)

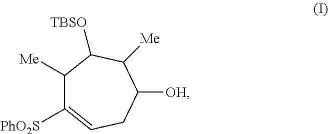

wherein the process comprising:

(a) treatment of a compound of formula (II)

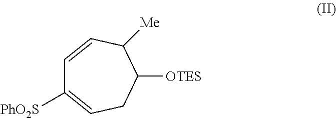

under an epoxidation reaction condition to prepare a compound of formula (III)

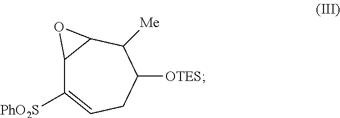

(b) treatment of said compound of formula (III) under a Lawton S_{N}2′ reaction condition with 3,5-dimethylpyrazole to prepare a compound of formula (IV)

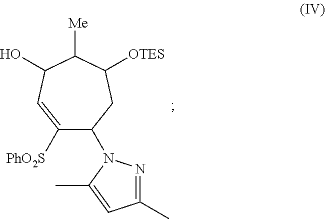

(c) Reacting said compound of formula (IV) with a Grignard reagent to prepare a compound of formula (V)

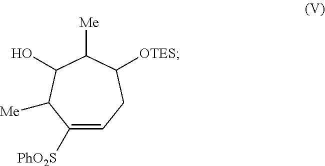

and

(d) silylation of said compound of formula (V) with TBSOTf, followed by a regioselective desilylation reaction to prepare the compound of formula (I).

In some embodiments, said epoxidation reaction condition of step (a) comprises oxone and solid NaHCO₃ in acetone. In some embodiments, said treatment of step (a) is carried out with vigorous mechanical stirring. In certain embodiments, said treatment of step (a) is carried out on a scale of about 23 g or above. In other embodiments, said treatment of step (a) is carried out on a scale of about 50 g or above. In some embodiments, said treatment of step (a) is carried out on a scale of about 75 g or above. In other embodiments, said treatment of step (a) is carried out on a scale of about 100 g or above. In some embodiments, said treatment of step (a) is followed by filtration on a silica pad for purification.

In other embodiments, said epoxidation reaction condition of step (a) comprises Jacobsen catalyst. In some embodiments, said treatment of step (a) is followed by the step of removal of Mn-Salen catalyst by adding 300% v/v hexanes, stirring at 0° C., and filtration on a celite pad. In some embodiments, the residue of said Mn-Salen catalyst is removed by addition of 0.5 equivalent of aqueous NaOCl. In certain embodiments, said treatment of step (a) is carried out on a scale of 50 g or above. In some embodiments, said treatment of step (a) is carried out on a scale of about 75 g or above. In other embodiments, said treatment of step (a) is carried out on a scale of about 100 g or above.

In some embodiments, said treatment of step (b) is carried out at a temperature of from about 23° C. to about 50° C. In other embodiments, said treatment of step (b) is followed by the step of purification by silica pad filtration. In certain embodiments, said treatment of step (b) is carried out on a scale of about 23 g or above. In other embodiments, said treatment of step (b) is carried out on a scale of about 50 g or above. In some embodiments, said treatment of step (b) is carried out on a scale of about 75 g or above. In other embodiments, said treatment of step (b) is carried out on a scale of about 100 g or above.

In some embodiments, said Grignard reagent of step (c) is MeMgBr. In some embodiments, said Grignard reagent of step (c) is added at a speed of 1-2 mL/min. In other embodiments, said Grignard reagent of step (c) is added at a speed of 1 mL/min. In certain embodiments, said Grignard reagent of step (c) is added at a speed of 2 mL/min. In certain embodiments, said Grignard reagent of step (c) is added at about 50° C. In some embodiments, said Grignard reagent of step (c) is added at about 25° C. In certain embodiments, said Grignard reagent of step (c) is added at a temperature of 0° C. In other embodiments, said Grignard reagent of step (c) is added at a temperature of below 0° C. In some embodiments, said reacting of step (c) is carried out on a scale of about 24 g or above. In other embodiments, said treatment of step (c) is carried out on a scale of about 50 g or above. In some embodiments, said treatment of step (c) is carried out on a scale of about 75 g or above. In other embodiments, said treatment of step (c) is carried out on a scale of about 100 g or above.

In some embodiments, said regioselective desilylation reaction of step (d) is carried out in the presence of CSA or TBAF. In other embodiments, said regioselective desilylation reaction of step (d) is carried out in the presence of CSA. In some embodiments, the product of said regioselective desilylation reaction of step (d) is purified by a short silica pad filtration.

In some embodiments, said the compound of formula (I) is represented by compounds 16, 2, or ent-2:

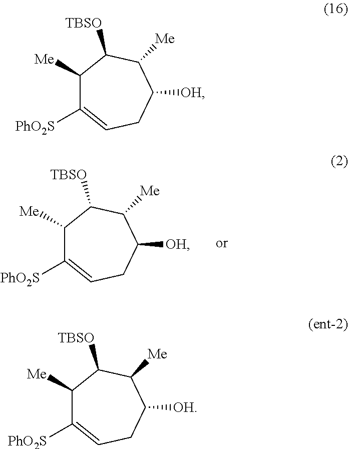

In some embodiments, said compound of formula (II) is prepared by

(a) treatment of a compound of formula (VI)

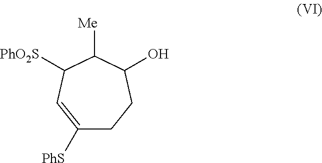

with TESCl and imidazole in an organic solvent to prepare a compound of formula (VII)

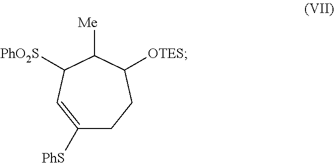

(b) treatment of said compound of formula (VII) under a desulfonylation reaction condition to prepare a compound of formula (VIII)

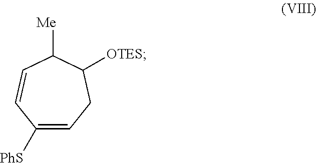

and

(c) treatment of said compound of formula (VIII) under an oxidation reaction condition to prepare the compound of formula (II).

In some embodiments, said organic solvent of step (a) is 1,2-dicholoroethane. In some embodiments, said treatment of step (a) is carried out at a temperature of from about 0° C. to about 23° C. In some embodiments, said treatment of step (a) is carried out at a temperature of from about 0° C. to about 50° C. In certain embodiments, said treatment of step (a) is carried out on a scale of about 87 g or above. In some embodiments, said treatment of step (a) is carried out on a scale of about 100 g or above. In some embodiments, said treatment of step (a) is followed by filtration and concentration for workup.

In some embodiments, said desulfonylation reaction condition of step (b) comprises AlMe₃ and a base. In some embodiments, said treatment of step (b) is carried out at about −78° C. In other embodiments, said treatment of step (b) is carried out at a temperature of from about −78° C. to about 23° C. In certain embodiments, said treatment of step (b) is carried out at a temperature of from about −78° C. to about 0° C. In some embodiments, said treatment of step (b) is carried out at a temperature of from about −78° C. to about 50° C. In some embodiments, said treatment of step (b) is followed by the step of transferring to a mechanically stirred mixture of aqueous HCl-crushed ice for workup.

In some embodiments, said oxidation reaction condition of step (c) is the Noyori oxidation condition. In certain embodiments, said treatment of step (c) is carried out on a scale of about 25 g or above. In other embodiments, said treatment of step (c) is carried out on a scale of about 60 g or above. In some embodiments, said treatment of step (c) is carried out on a scale of about 75 g or above. In other embodiments, said treatment of step (c) is carried out on a scale of about 100 g or above. In some embodiments, said treatment of step (c) is carried out at a temperature of from about 0° C. to about 10° C. In some embodiments, said treatment of step (c) is followed by short silica pad filtration for workup.

In some embodiments, said compound of formula (II) is represented by compounds 10, 22, or ent-22:

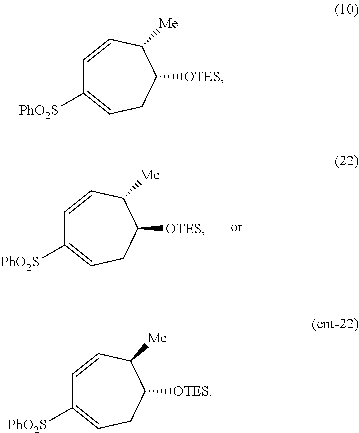

In some embodiments, said compound of formula (VI) is prepared by treatment of a compound of formula (IX)

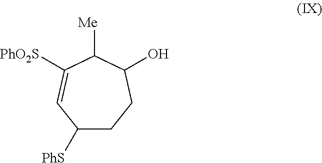

with DBU in acetonitrile.

In certain embodiments, said compound of formula (VI) is prepared on a scale of about 65 g or above.

In some embodiments, said compound of formula (VI) is prepared by treatment of a compound of formula (X)

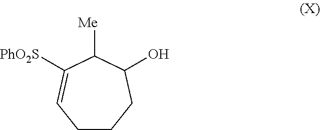

with NaHMDS in THF, followed by addition of PhSSPh.

In some embodiments, said treatment is carried out at a temperature of from about −78° C. to about 23° C. In certain embodiments, said compound of formula (VI) is prepared on a scale of about 80 g or above. In other embodiments, said compound of formula (VI) is prepared on a scale of about 100 g or above.

The present invention provides a process for preparing a compound of formula (II)

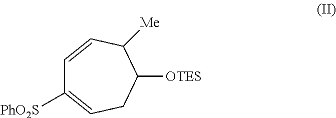

the process comprising treatment of a compound of formula (VII)

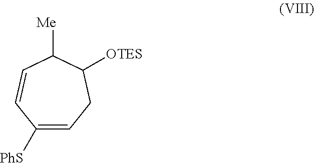

under an oxidation reaction condition.

In some embodiments, said oxidation reaction condition is the Noyori oxidation condition. In certain embodiments, said treatment is carried out on a scale of about 25 g or above. In other embodiments, said treatment is carried out on a scale of about 60 g or above. In some embodiments, said treatment is carried out on a scale of about 75 g or above. In other embodiments, said treatment is carried out on a scale of about 100 g or above.

In some embodiments, said treatment is carried out at a temperature of from about 0° C. to about 10° C. In some embodiments, said treatment is carried out at a temperature of from about 0° C. to about 25° C. In certain embodiments, said treatment is followed by short silica pad filtration for workup.

In some embodiments, said compound of formula (II) is represented by compounds 10, 22, and ent-22:

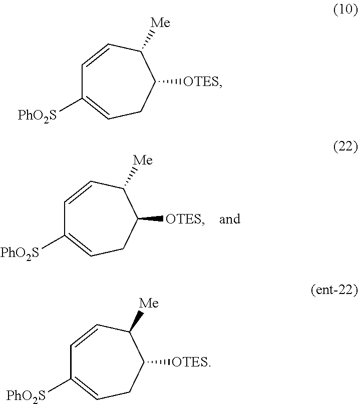

In some embodiments, said compound of formula (VIII) is prepared by treatment of a compound of formula (VII)

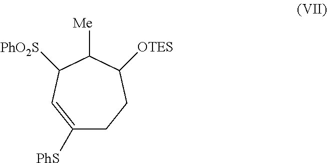

under a desulfonylation reaction condition.

In some embodiments, said desulfonylation reaction condition comprises AlMe₃ and a base. In some embodiment, said treatment is carried out at about −78° C. In other embodiments, said treatment is carried out at a temperature of from about −78° C. to about 0° C. In certain embodiments, said treatment is carried out at a temperature of from about −78° C. to about 23° C. In some embodiments, said treatment is followed by the step of transferring to a mechanically stirred mixture of aqueous HCl-crushed ice for workup.

In some embodiments, said compound of formula (VII) is prepared by treatment of a compound of formula (VI)

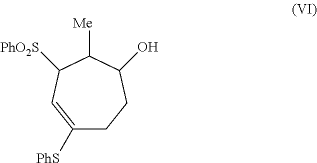

with TESCl and imidazole in an organic solvent.

In some embodiments, said organic solvent is 1,2-dicholoroethane. In some embodiments, said treatment is carried out at a temperature of from about 0° C. to about 23° C. In certain embodiments, said treatment is carried out on a scale of about 87 g or above. In other embodiments, said treatment is carried out on a scale of about 100 g or above. In some embodiments, said treatment is followed by filtration and concentration for workup.

In some embodiments, said compound of formula (VI) is prepared by treatment of a compound of formula (IX)

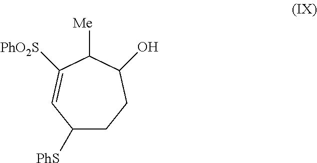

with DBU in acetonitrile.

In some embodiments, said compound of formula (VI) is prepared on a scale of about 65 g or above. In some embodiments, said compound of formula (VI) is prepared on a scale of about 100 g or above.

In other embodiments, said compound of formula (VI) is prepared by treatment of a compound of formula (X)

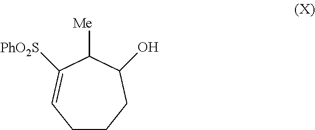

with NaHMDS in THF, followed by addition of PhSSPh.

In some embodiments, said treatment is carried out at a temperature of from about −78° C. to about 23° C. In certain embodiments, the compound of formula (VI) is prepared on a scale of about 80 g or above. In certain embodiments, the compound of formula (VI) is prepared on a scale of about 100 g or above.

In yet another aspect, the present invention provides a process for preparing compound (16)

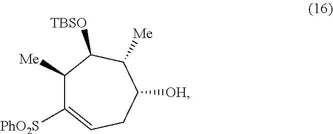

the process comprising:

(d) treatment of compound (10)

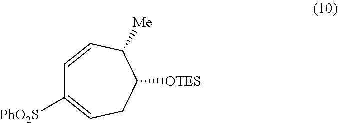

under an epoxidation reaction condition to prepare compound (13)

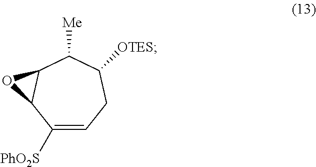

(e) treatment of said compound (13) under a Lawton S_{N}2′ reaction condition with 3,5-dimethylpyrazole to prepare compound (14)

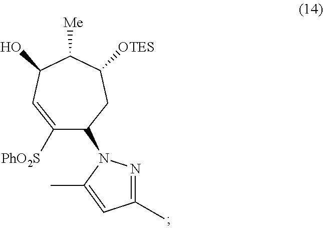

(f) Reacting said compound (14) with a Grignard reagent to prepare compound (15)

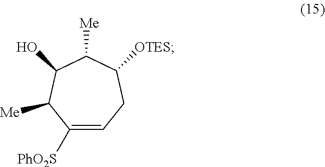

and

(d) silylation of said compound (15) with TBSOTf, followed by a regioselective desilylation reaction to prepare compound (16).

In some embodiments, said epoxidation reaction condition of step (a) comprises oxone and solid NaHCO₃ in acetone. In some embodiments, said treatment of step (a) is carried out with vigorous mechanical stirring. In certain embodiments, said treatment of step (a) is carried out on a scale of about 23 g or above. In other embodiments, said treatment of step (a) is carried out on a scale of about 50 g or above. In some embodiments, said treatment of step (a) is carried out on a scale of about 75 g or above. In certain embodiments, said treatment of step (a) is carried out on a scale of about 100 g or above. In some embodiments, said treatment of step (a) is followed by filtration on a silica pad for purification.

In some embodiments, said treatment of step (b) is carried out at a temperature of from about 23° C. to about 50° C. In some embodiments, said treatment of step (b) is carried out at a temperature of from about 0° C. to about 50° C. In some embodiments, said treatment of step (b) is followed by the step of purification by silica pad filtration. In certain embodiments, said treatment of step (b) is carried out on a scale of about 23 g or above. In certain embodiments, said treatment of step (b) is carried out on a scale of about 50 g or above. In other embodiments, said treatment of step (b) is carried out on a scale of about 75 g or above. In some embodiments, said treatment of step (b) is carried out on a scale of about 100 g or above.

In some embodiments, said Grignard reagent of step (c) is MeMgBr. In some embodiments, said Grignard reagent of step (c) is added at a speed of 1-2 mL/min. In some embodiments, said Grignard reagent of step (c) is added at about 50° C. In some embodiments, said Grignard reagent of step (c) is added at about 25° C. In some embodiments, said Grignard reagent of step (c) is added at about 0° C. In certain embodiments, said reacting of step (c) is carried out on a scale of about 24 g or above. In some embodiments, said reacting of step (c) is carried out on a scale of about 50 g or above. In some embodiments, said reacting of step (c) is carried out on a scale of about 75 g or above. In some embodiments, said reacting of step (c) is carried out on a scale of about 100 g or above.

In some embodiments, said regioselective desilylation reaction of step (d) is carried out in the presence of CSA or TBAF. In some embodiments, said regioselective desilylation reaction of step (d) is carried out in the presence of CSA. In some embodiments, the product of said regioselective desilylation reaction of step (d) is purified by a short silica pad filtration.

In some embodiments, said compound (10) is prepared by

(a) treatment of compound (7)

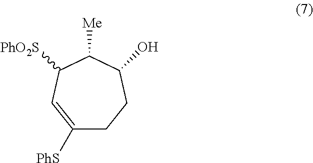

with TESCl and imidazole in an organic solvent to prepare compound (8)

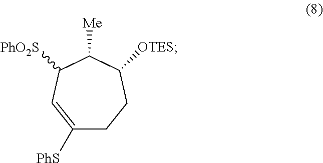

(b) treatment of said compound (8) under a desulfonylation reaction condition to prepare compound (9)

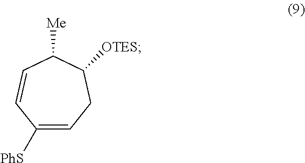

and

(c) treatment of said compound (9) under an oxidation reaction condition to prepare compound (10).

In some embodiments, said organic solvent of step (a) is 1,2-dicholoroethane. In some embodiments, said treatment of step (a) is carried out at a temperature of from about 0° C. to about 23° C. In some embodiments, said treatment of step (a) is carried out at a temperature of from about 0° C. to about 50° C. In certain embodiments, said treatment of step (a) is carried out on a scale of about 65 g or above. In other embodiments, said treatment of step (a) is carried out on a scale of about 100 g or above. In some embodiments, said treatment of step (a) is followed by filtration and concentration for workup.

In some embodiments, said desulfonylation reaction condition of step (b) comprises AlMe₃ and a base. In certain embodiments, said treatment of step (b) is carried out at about −78° C. In other embodiments, said treatment of step (b) is carried out at a temperature of from about −78° C. to about 23° C. In certain embodiments, said treatment of step (b) is carried out at a temperature of from about −78° C. to about 0° C. In some embodiments, said treatment of step (b) is followed by the step of transferring to a mechanically stirred mixture of aqueous HCl-crushed ice for workup.

In some embodiments, said oxidation reaction condition of step (c) is the Noyori oxidation condition. In certain embodiments, said treatment of step (c) is carried out on a scale of about 25 g or above. In other embodiments, said treatment of step (c) is carried out on a scale of about 50 g or above. In some embodiments, said treatment of step (c) is carried out on a scale of about 75 g or above. In certain embodiments, said treatment of step (c) is carried out on a scale of about 100 g or above. In some embodiments, said treatment of step (c) is carried out at a temperature of from about 0° C. to about 10° C. In some embodiments, said treatment of step (c) is followed by short silica pad filtration for workup.

In some embodiments, said compound (7) is prepared by treatment of compound (6)

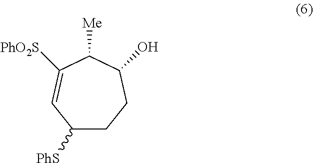

with DBU in acetonitrile.

In certain embodiments, said compound (6) is prepared on a scale of about 65 g or above. In certain embodiments, said compound (6) is prepared on a scale of about 100 g or above.

In yet another aspect, the present invention provides a process for preparing compound (2)

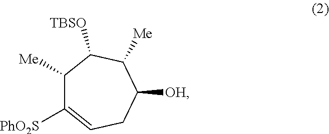

the process comprising:

(e) treatment of compound (22)

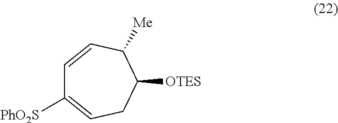

under an epoxidation reaction condition to prepare compound (25-OTES)

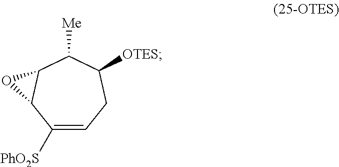

(f) treatment of said compound (25-OTES) under a Lawton S_{N}2′ reaction condition with 3,5-dimethylpyrazole to prepare compound (27)

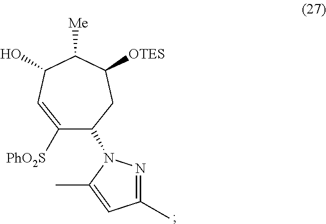

(g) Reacting said compound (27) with a Grignard reagent to prepare compound (28a)

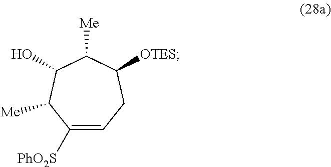

and

(d) silylation of said compound (28a) with TBSOTf, followed by a regioselective desilylation reaction to prepare compound (2).

In some embodiments, said epoxidation reaction condition of step (a) comprises Jacobsen catalyst. In some embodiments, said treatment of step (a) is followed by the step of removal of Mn-Salen catalyst by adding 300% v/v hexanes, stirring at 0° C., and filtration on a celite pad. In certain embodiments, the residue of said Mn-Salen catalyst is removed by addition of 0.5 equivalent of aqueous NaOCl. In some embodiments, said treatment of step (a) is carried out on a scale of 50 g or above. In some embodiments, said treatment of step (a) is carried out on a scale of 75 g or above. In some embodiments, said treatment of step (a) is carried out on a scale of 100 g or above.

In some embodiments, said treatment of step (b) is carried out at a temperature of from about 23° C. to about 50° C. In some embodiments, said treatment of step (b) is carried out at a temperature of from about 0° C. to about 50° C. In some embodiments, said treatment of step (b) is carried out at a temperature of from about 0° C. to about 23° C. In some embodiments, said treatment of step (b) is followed by the step of purification by silica pad filtration. In certain embodiments, said treatment of step (b) is carried out on a scale of about 50 g or above. In other embodiments, said treatment of step (b) is carried out on a scale of about 75 g or above. In some embodiments, said treatment of step (b) is carried out on a scale of about 100 g or above.

In some embodiments, said Grignard reagent of step (c) is MeMgBr. In certain embodiments, said Grignard reagent of step (c) is added at a speed of 1-2 mL/min. In some embodiments, said Grignard reagent of step (c) is added at a speed of 1 mL/min. In certain embodiments, said Grignard reagent of step (c) is added at a speed of 2 mL/min. In some embodiments, said Grignard reagent of step (c) is added at about 50° C. In some embodiments, said Grignard reagent of step (c) is added at about 25° C. In some embodiments, said Grignard reagent of step (c) is added at about 0° C.

In some embodiments, said regioselective desilylation reaction of step (d) is carried out in the presence of CSA or TBAF. In some embodiments, said regioselective desilylation reaction of step (d) is carried out in the presence of CSA. In some embodiments, the product of said regioselective desilylation reaction of step (d) is purified by a short silica pad filtration.

In some embodiments, said compound (22) is prepared by

(a) treatment of compound (19)

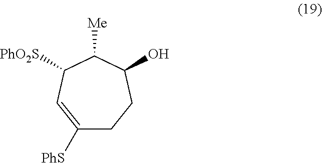

with TESCl and imidazole in an organic solvent to prepare compound (20)

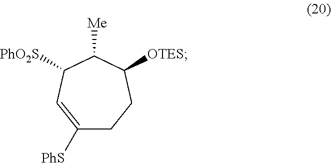

(b) treatment of said compound (20) under a desulfonylation reaction condition to prepare compound (21)

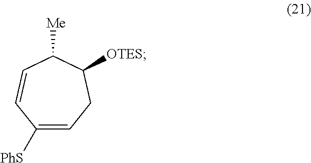

and

(c) treatment of said compound (21) under an oxidation reaction condition to prepare compound (22).

In some embodiments, said organic solvent of step (a) is 1,2-dicholoroethane. In some embodiments, said treatment of step (a) is carried out at a temperature of from about 0° C. to about 23° C. In some embodiments, said treatment of step (a) is carried out at a temperature of from about 0° C. to about 50° C. In some embodiments, said treatment of step (a) is carried out at a temperature of from about 23° C. to about 50° C. In certain embodiments, said treatment of step (a) is carried out on a scale of about 67 g or above. In other embodiments, said treatment of step (a) is carried out on a scale of about 100 g or above. In some embodiments, said treatment of step (a) is followed by filtration and concentration for workup.

In some embodiments, said desulfonylation reaction condition of step (b) comprises AlMe₃ and a base. In some embodiments, said treatment of step (b) is carried out at about −78° C. In other embodiments, said treatment of step (b) is carried out at a temperature of from about −78° C. to about 23° C. In some embodiments, said treatment of step (b) is carried out at a temperature of from about −78° C. to about 0° C. In certain embodiments, said treatment of step (b) is carried out on a scale of about 87 g or above. In certain embodiments, said treatment of step (b) is carried out on a scale of about 100 g or above. In some embodiments, said treatment of step (b) is followed by the step of transferring to a mechanically stirred mixture of aqueous HCl-crushed ice for workup.

In some embodiments, said oxidation reaction condition of step (c) is the Noyori oxidation condition. In some embodiments, said treatment of step (c) is carried out on a scale of about 61 g or above. In some embodiments, said treatment of step (c) is carried out on a scale of about 75 g or above. In some embodiments, said treatment of step (c) is carried out on a scale of about 100 g or above. In some embodiments, said treatment of step (c) is carried out at a temperature of from about 0° C. to about 10° C. In some embodiments, said treatment of step (c) is carried out at a temperature of from about 0° C. to about 25° C. In some embodiments, said treatment of step (c) is carried out at a temperature of from about 0° C. to about 50° C. In certain embodiments, said treatment of step (c) is followed by short silica pad filtration for workup.

In some embodiments, said compound (19) is prepared by treatment of compound (18a)

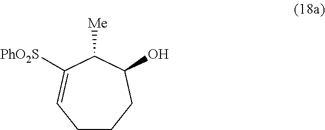

with NaHMDS in THF at a temperature of from about −78° C. to about 23° C., followed by addition of PhSSPh.

In some embodiments, said compound (19) is prepared on a scale of about 80 g or above. In some embodiments, said compound (19) is prepared on a scale of about 100 g or above.

In yet another aspect, the present invention provides a process for preparing compound (ent-2)

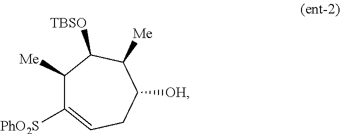

the process comprising:

(e) treatment of compound (ent-22)

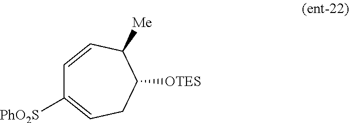

under an epoxidation reaction condition to prepare compound (ent-25-OTES)

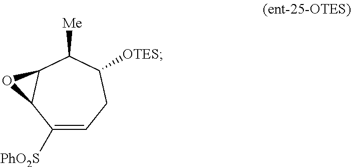

(f) treatment of said compound (ent-25-OTES) under a Lawton S_{N}2′ reaction condition with 3,5-dimethylpyrazole to prepare compound (ent-27)

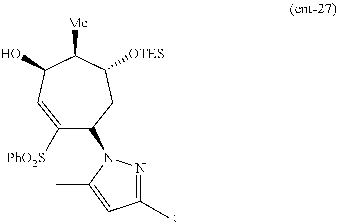

(g) Reacting said compound (ent-27) with a Grignard reagent to prepare compound (ent-28a)

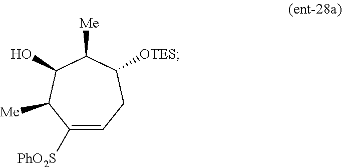

and

(h) silylation of said compound (ent-28a) with TBSOTf, followed by a regioselective desilylation reaction to prepare compound (ent-2).

In some embodiments, said epoxidation reaction condition of step (a) comprises Jacobsen catalyst. In some embodiments, said treatment of step (a) is followed by the step of removal of Mn-Salen catalyst by adding 300% v/v hexanes, stirring at 0° C., and filtration on a celite pad. In certain embodiments, the residue of said Mn-Salen catalyst is removed by addition of 0.5 equivalent of aqueous NaOCl. In some embodiments, said treatment of step (a) is carried out on a scale of 71 g or above. In some embodiments, said treatment of step (a) is carried out on a scale of 100 g or above.

In some embodiments, said treatment of step (b) is carried out at a temperature of from about 23° C. to about 50° C. In some embodiments, said treatment of step (b) is carried out at a temperature of from about 0° C. to about 50° C. In some embodiments, said treatment of step (b) is carried out at a temperature of from about 0° C. to about 23° C. In certain embodiments, said treatment of step (b) is followed by the step of purification by silica pad filtration.

In some embodiments, said Grignard reagent of step (c) is MeMgBr. In certain embodiments, said Grignard reagent of step (c) is added at a speed of 1-2 mL/min. In some embodiments, said Grignard reagent of step (c) is added at a speed of 1 mL/min. In other embodiments, said Grignard reagent of step (c) is added at a speed of 2 ml/min. In some embodiments, said Grignard reagent of step (c) is added at about 50° C. In some embodiments, said Grignard reagent of step (c) is added at about 0° C. In some embodiments, said Grignard reagent of step (c) is added at about 25° C.

In some embodiments, said regioselective desilylation reaction of step (d) is carried out in the presence of CSA or TBAF. In some embodiments, said regioselective desilylation reaction of step (d) is carried out in the presence of CSA. In some embodiments, the product of said regioselective desilylation reaction of step (d) is purified by a short silica pad filtration.

In some embodiments, said compound (ent-22) is prepared by

(a) treatment of compound (ent-19)

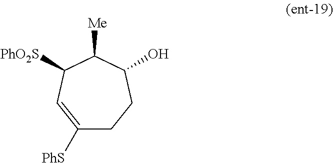

with TESCl and imidazole in an organic solvent to prepare compound (ent-20)

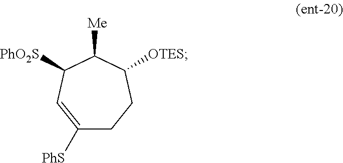

(b) treatment of said compound (ent-20) under a desulfonylation reaction condition to prepare compound (ent-21)

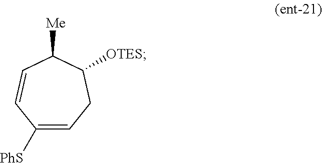

and

(c) treatment of said compound (ent-21) under an oxidation reaction condition to prepare compound (ent-22).

In some embodiments, said organic solvent of step (a) is 1,2-dicholoroethane. In some embodiments, said treatment of step (a) is carried out at a temperature of from about 0° C. to about 23° C. In some embodiments, said treatment of step (a) is carried out at a temperature of from about 0° C. to about 50° C. In some embodiments, said treatment of step (a) is carried out at a temperature of from about 23° C. to about 50° C. In certain embodiments, said treatment of step (a) is carried out on a scale of about 67 g or above. In other embodiments, said treatment of step (a) is carried out on a scale of about 100 g or above. In some embodiments, said treatment of step (a) is followed by filtration and concentration for workup.

In some embodiments, said desulfonylation reaction condition of step (b) comprises AlMe₃ and a base, for example, Hünigs base.

In some embodiments, said treatment of step (b) is carried out at about −78° C. In other embodiments, said treatment of step (b) is carried out at a temperature of from about −78° C. to about 23° C. In other embodiments, said treatment of step (b) is carried out at a temperature of from about −78° C. to about 0° C. In certain embodiments, said treatment of step (b) is carried out on a scale of about 87 g or above. In other embodiments, said treatment of step (b) is carried out on a scale of about 100 g or above. In some embodiments, said treatment of step (b) is followed by the step of transferring to a mechanically stirred mixture of aqueous HCl-crushed ice for workup.

In some embodiments, said oxidation reaction condition of step (c) is the Noyori oxidation condition. In certain embodiments, said treatment of step (c) is carried out on a scale of about 61 g or above. In other embodiments, said treatment of step (c) is carried out on a scale of about 75 g or above. In some embodiments, said treatment of step (c) is carried out on a scale of about 100 g or above. In some embodiments, said treatment of step (c) is carried out at a temperature of from about 0° C. to about 10° C. In some embodiments, said treatment of step (c) is carried out at a temperature of from about 0° C. to about 25° C. In some embodiments, said treatment of step (c) is carried out at a temperature of from about 10° C. to about 25° C. In some embodiments, said treatment of step (c) is followed by short silica pad filtration for workup.

In some embodiments, said compound (ent-19) is prepared by treatment of compound (ent-18a)

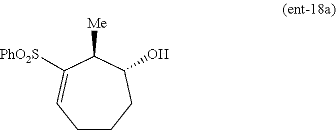

with NaHMDS in THF at a temperature of from about −78° C. to about 23° C., followed by addition of PhSSPh.

The present invention provides processes to access key stereotetrads 16, 2, and ent-2 as demonstrated in Scheme 1.

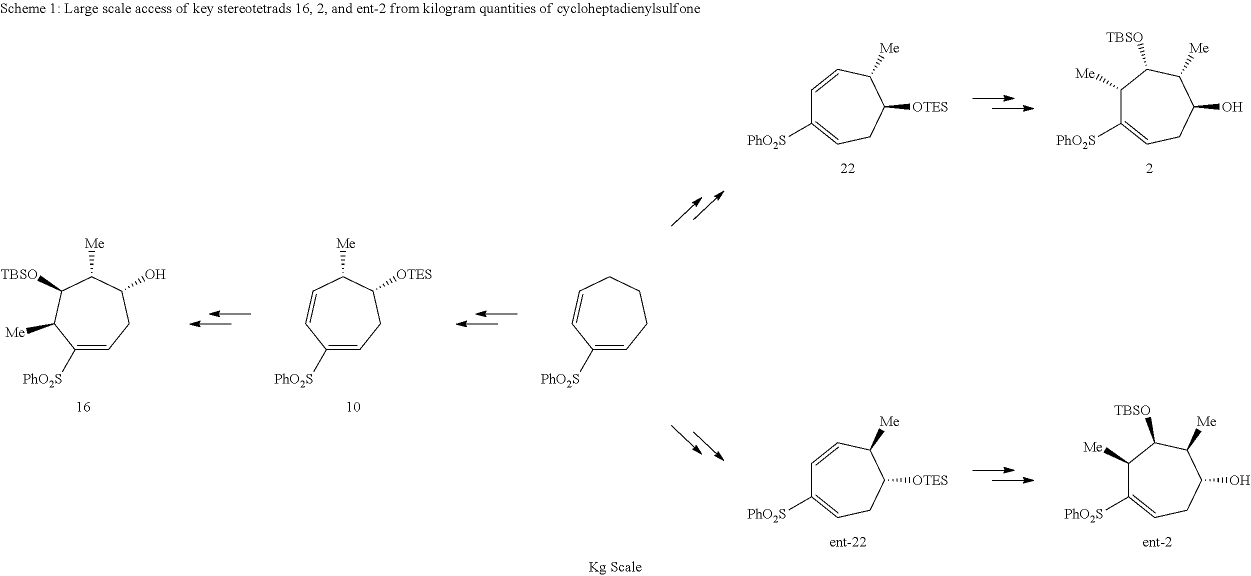

### Synthesis of Compound (10) by an Improved Process

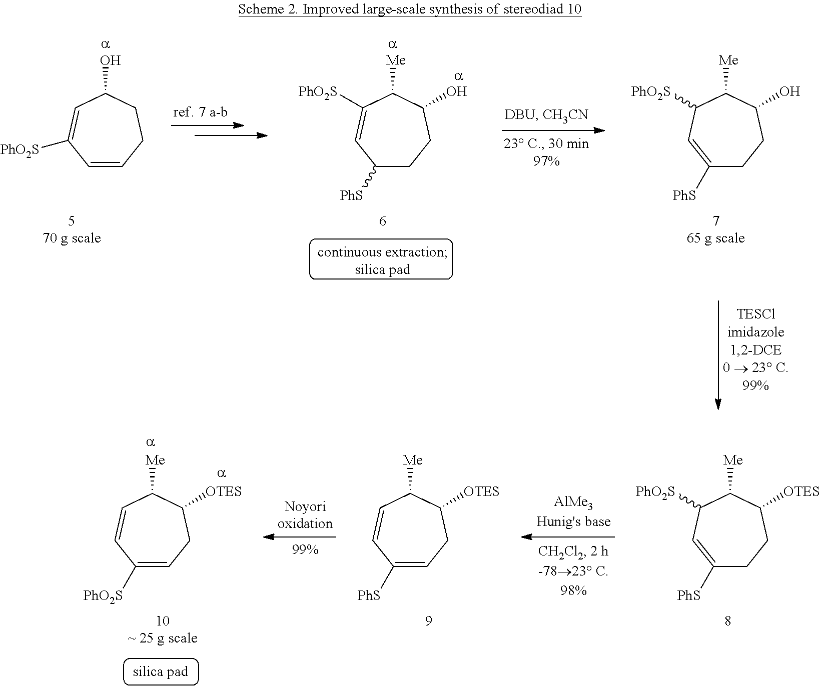

Dienylsulfone alcohol 5 was transformed to vinylsulfone 6 on 70 g scale via a syn-directed methylation-sulfenylation one pot sequence. Residual benzenethiol was removed via continuous extraction of a biphasic CH₃CN-hexane system. Equilibration of 6 to allylsulfone 7 was best achieved employing catalytic DBU in acetonitrile instead of toluene. The reaction proceeds slowly in toluene and requires heating due to partial insolubility. In contrast, the reaction is complete in 30 minutes in acetonitrile at room temperature (Table 1).

Residual benzenethiol interferes with this equilibration, suggesting that benzenethiolate anion is not capable of effecting the equilibration even at 90° C. Crude 7 was subjected to silylation employing TESCl and imidazole in 1,2-DCE, the workup being only filtration and concentration of 8. Crude 8 was sufficiently pure to be subjected to desulfonylation employing AlMe₃ and Hünigs base. In contrast to the previous method, silylation prior to desulfonylation offered a cleaner conversion requiring one less equivalent of AlMe₃. The addition of AlMe₃ is exothermic and must be executed at −78° C. while the desired elimination does not commence before warming to 25° C. Aqueous acidic workup in crushed ice efficiently removed the aluminum residues without affecting the TESO group, thus quantitatively providing 9. Finally, Noyori oxidation of 9 was optimized for large scale (25 g) where the TESO group once again survived the presence of hydrogen peroxide. While the Noyori oxidation (Na₂WO₄, PhP(O)(OH)₂, oct₃MeNHSO₄, H₂O₂) does not proceed below 10° C., addition of hydrogen peroxide must be performed at 0° C. to avoid exothermic frothing, followed by removal of the ice bath.

In contrast, large-scale reactions require slow warming without ice bath removal to avoid a sudden exothermic onset and possible eruptions. Silica pad filtration provided highly pure 10 as a yellow oil on scales up to 25 g (scheme 2).

Solvent Effects in Equilibration of Vinylsulfone 6 to Allylsulfone 7 were observed. 6 is received as an inseparable mixture of diastereomers. However, equilibration with base afforded 7 as two diastereomeric allylsulfones (7a and 7b) separable by column chromatography. A summary of solvent effects observed in the equilibration of 6 to 7 is represented in Table 1.

TABLE 1
Solvent screening in equilibration of 6 to 7

| Entry | Solvent | ε | DBU loading | Molarity | Temp. | Time | |
|---|---|---|---|---|---|---|---|
| 1 | toluene | 2.4 | 1 mol % | [0.1M]^{a} | 90° C. | 1-2 | h |
| 2 | toluene | 2.4 | 5 mol % | [0.1M]^{a} | 25° C. | 12 | h |
| 3 | CHCl₃ | 4.8 | 5 mol % | [0.4M] | 25° C. | 30 | min^{b} |
| 4 | CH₂Cl₂ | 9.1 | 5 mol % | [0.4M] | 25° C. | 30 | min^{b} |
| 5 | CH₃CN | 37.5 | 5 mol % | [1.0M] | 25° C. | 30 | min^{b} |

^{a}precipitation from reaction occurs at concentrations > [0.1M].
^{b}reaction is instantaneous (1-5 min) on small scales.

The stereochemistry of 7b (lower R_{f} diastereomer) was determined as all syn via crystal structure determination of acetate derivative 12.

Deconjugation of vinylsulfones to allylsulfones was found to be highly stereospecific. The stereochemistry of the resulting allylsulfone is dictated by the relative stereochemistry between the neighboring hydroxyl and methyl groups. Moreover, the type of base employed, and the mode of anion quench (kinetic vs. thermodynamic) significantly affect the stereochemical outcome of this conversion. While anti relationship always dictates syn sulfonyl group relative to the methyl (Table 2, entries, 1&2), a syn relationship had variable outcomes depending on the conditions employed. For example (treating vinylsulfone mixture 6 with DBU in acetonitrile always provided a diastereomeric mixture of 2.6:1 ratio (thermodynamic; Table 2, entry 4).

However, when dienyl alcohol 5 was treated with MeMgBr followed by kinetic quenching at −78° C., a single diastereomer was obtained as determined by ¹H NMR (Table 2, entry 3). A summary of factors affecting deconjugation of vinylsulfones to allylsulfones is presented in Table 2.

TABLE 2
Factors affecting deconjugation of vinylsulfones to allylsulfones

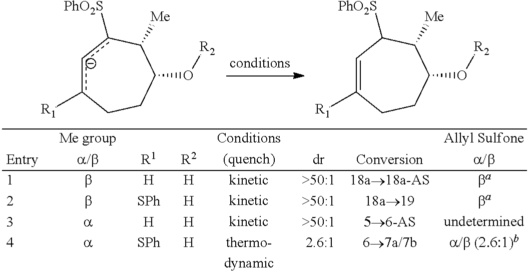

^{a}stereochemistry determined by X-ray crystal structure(s).
^{b}stereochemistry of α-isomer derivative 12 was determined by X-ray
crystal structure.

The higher R_{f} diastereomer 7a was found to undergo facile photocyclization (Photocyclization of 7a occurred over two weeks at 23° C., room light) to 11. Photocyclization of 7a occurred over two weeks at 23° C., room light. Cyclization of 7a did not proceed in the dark even after reflux at 110° C. for 48 h with quantitative recovery of the starting material. In contrast, 7b did not suffer cyclization under photo, radical or thermal conditions (Scheme 3).

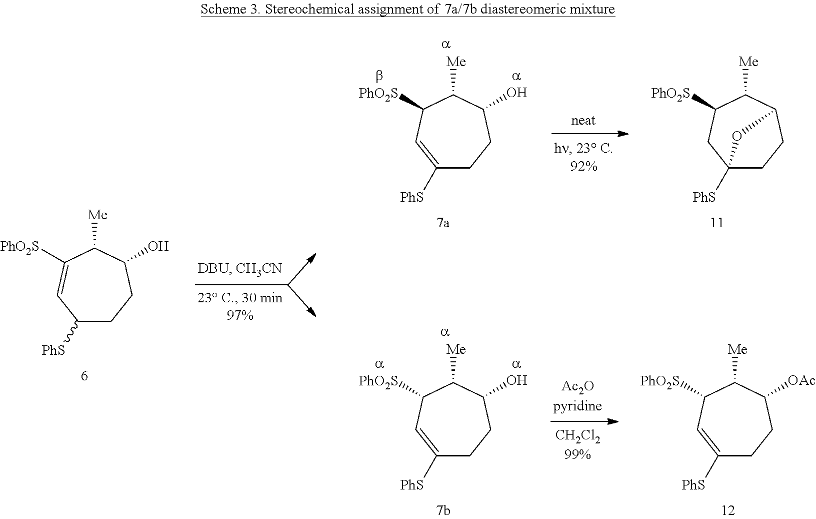

While hydroxyallylsulfone 7a underwent photocyclization to oxolane 11, 7b failed to cyclize. This difference in reactivity is solely dictated by the absolute stereochemistry of the allylsulfone group. The phenylsulfone moiety is poised in a β-pseudo-equatorial position in 7a forcing the ring convexity towards the α-face and positioning the α-hydroxyl group in close proximity to the vinylsulfone olefin thus facilitating cyclization. In contrast, the phenylsulfone moiety in 7b assumes a distorted α-pseudo-axial geometry where the ring is stabilized in a pseudo-chair conformation while the α-hydroxyl group is in perfect pseudo-axial position far away from the vinylsulfone olefin. The calculated distances between the α-hydroxyl group and the sp² carbon bearing the phenylsulfide is 3.0 A° for 7a and 3.7 A° for 7b as calculated from MM2 minimization of both structures.

Substrate directed epoxidation of 10 employing oxone/acetone did not affect the TESO group at 23° C., and afforded 13 as a single diastereomer. It was observed that portion-wise addition of oxone (4 doses at equal intervals) effected completion as compared to adding the oxidant in a single portion. The reaction did not warm beyond 35° C. regardless of the scale. Vigorous mechanical stirring is crucial due to the presence of solid NaHCO₃ which is necessary to prevent desilylation by the acidic oxone.

Lawton S_{N}2′ reaction of 13 with 3,5-Dimethylpyrazole (DMP) occurs at 23° C. but does not go to completion below 50° C. However, extended reaction times result in partial desilylation to diol 14 (Scheme 4). Subsequent syn-directed methylation proved to be extremely sensitive to the purity of 14. Therefore, a silica pad filtration is a “must” at this stage, yielding 75-80% of 14 over two steps.

The syn-directed methylation of 14 was not a trivial transformation. It was observed that the reaction proceeds poorly at 0° C., and only 50% conversion was achieved when Grignard reagent was added at 23° C. due to a temporary exotherm that ceases despite the presence of unconsumed 14. Attempts to add more MeMgBr and/or warming the reaction resulted in consumption of 15 in a subsequent conjugate addition of the Grignard reagent in preference to addition to 14. No improvement was observed when MeMgBr and 14 were simultaneously added to a third vessel. After extensive investigation, it was discovered that the initial exotherm must be maintained for an uninterrupted reaction. Indeed, dropwise addition of MeMgBr to a solution of 14 at 50° C. resulted in a clean conversion to 15 with neither unreacted 14 nor over-methylation of 15 being observed. This experiment was also very practical on multigram scales with controlled Grignard reagent addition 1-2 mL/min employing a liquid addition pump since employing syringes with these scales is impractical.

Silylation of 15 with TBSOTf followed by regioselective desilylation of the TESO-group employing CSA or TBAF was scalable as a one pot procedure. A short silica pad filtration furnished stereotetrad 16 in 80% yield as a single diastereomer (Scheme 4).

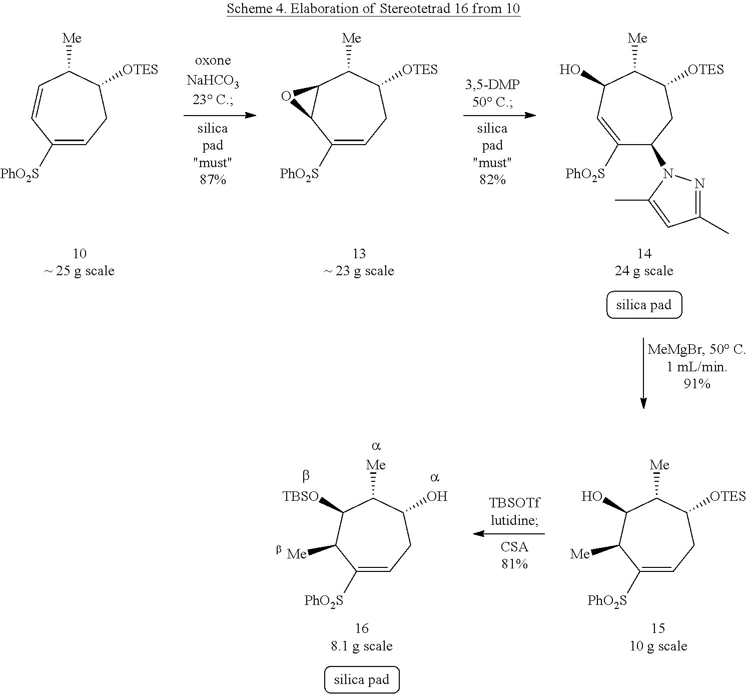

### Synthesis of Compound (22) (Stereodiad 22) by an Improved Process

While syn-methylation of dienyl alcohol 6 to stereodiad 7 is facilitated and directed by the presence of a free alcohol moiety, anti-methylation of 17 was not easily achieved.

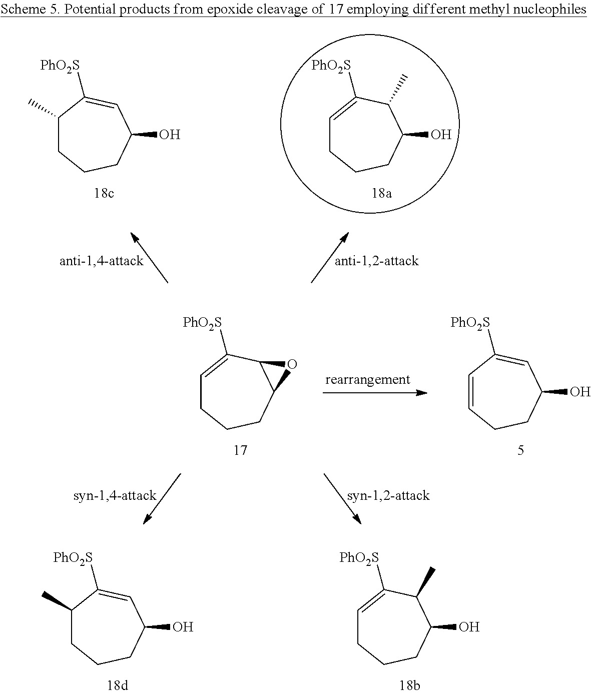

Treating 17 with methyl nucleophiles led to five different products depending on the reaction conditions (Scheme 5). However, treating 17 with AlMe₃ (2-3 equivalents) initially gave the most promising results (Table 3).

TABLE 3
Cleavage of epoxide 17 with different organometallic reagents

| Entry | Reagent | Equivalent | Result |
|---|---|---|---|
| 1 | MeLi | 1 | rearrangement to 5 |
| 2 | MeMgBr | 1-3 | no reaction |
| 3 | Me₂Zn | 1-3 | no reaction |
| 4 | AlMe₃ | 1 | decomposition |
| 5 | AlMe₃ | 2 | major 18a |
| 6 | AlMe₃ | 3 | major 18a |

### Trimethylaluminum Data

Further experimentation revealed solvent and concentration effects in the methylation of epoxide 17 employing commercially available AlMe₃ in hexanes. The diastereoselectivity in favor of 18a (anti-1,2-attack) increased proportionally with concentrations [0.05M] to [0.4M]. Moreover, at any particular concentration, diastereoselectivity in favor of 18a was greater in dichloromethane than it is in toluene (Table 4). The overall dielectric constant (DEC) in dichloromethane reaction is inversely proportional to the reaction concentration.

TABLE 4
Solvent and concentration effects in
AlMe₃-mediated anti-methylation of epoxide 17

| | | | | DEC | | DEC | |
|---|---|---|---|---|---|---|---|
| | | | Total volume | (DCM) | Yield | (Toluene) | Yield |
| [0.05M] | | | | | | | |
| solvent | AlMe₃ | Hexanes | 19.75 mL | 8.64 | 60% | 2.37 | 49% |
| 18.5 mL | 0.3 mL | 0.95 mL | | | | | |
| (93.67%) | | (6.33%) | | | | | |
| [0.10M] | | | | | | | |
| solvent | AlMe₃ | Hexanes | 9.75 mL | 8.18 | 56% | 2.33 | 42% |
| 8.5 mL | 0.3 mL | 0.95 mL | | | | | |
| (87.18%) | | (12.82%) | | | | | |
| [0.20M] | | | | | | | |
| solvent | AlMe₃ | Hexanes | 4.75 mL | 7.20 | 83% | 2.27 | 60% |
| 3.5 mL | 0.3 mL | 0.95 mL | | | | | |
| (73.68%) | | (26.32%) | | | | | |
| [0.30M] | | | | | | | |
| solvent | AlMe₃ | Hexanes | 3.05 mL | 6.15 | 68% | 2.19 | 58% |
| 1.8 mL | 0.3 mL | 0.95 mL | | | | | |
| (59.02%) | | (40.98%) | | | | | |
| [0.40M] | | | | | | | |
| solvent | AlMe₃ | Hexanes | 2.25 mL | 5.09 | 89% | 2.17 | 91% |
| 1.0 mL | 0.3 mL | 0.95 mL | | | | | |
| (44.44%) | | (55.56%) | | | | | |
| [0.50M] | | | | | | | |
| solvent | AlMe₃ | Hexanes | 1.75 mL | 3.95 | 78% | 2.04 | 74% |
| 0.5 mL | 0.3 mL | 0.95 mL | | | | | |
| (28.57%) | | (71.43%) | | | | | |

DEC (Dielectric constant); DEC of DCM 9.1, DEC toluene 2.4, DEC hexanes 1.88; empirical calculation of overall DEC=[solvent % (DEC)+hexane % (1.88)]; the tabulated yields are based on ¹H NMR ratio of 18a:18b of the crude reactions since 18a and 18b are inseparable by flash column chromatography.

In toluene reactions overall DEC does not effect significant changes in DEC according to the empirical formula employed in Table 4. The calculated values for the overall DEC become closest for both dichloromethane and toluene reactions at [0.4M] and [0.5M] concentrations, and this might explain the close dr in both solvents at these concentrations. The best dr 12.5:1 was obtained with both dichloromethane and toluene at [0.4M].

### Aluminoxane Data.

In contrast to methyl lithium MeLi, methylmagnesium bromide MeMgBr (Grignard reagent), or dimethyl zinc Me₂Zn, addition of water H₂O does not quench trimethylaluminum AlMe₃ but rather forms other active methylating species called “aluminoxanes” that contain Al—O—Al bonds.

Aluminoxanes can be more powerful in epoxide cleavages than AlMe₃ due to higher order complexation to the epoxide. Adding different water ratios relative to AlMe₃ gave different dr favoring the desired anti 18a. In general, a ratio of 5:1 (AlMe₃: H₂O) was required for best dr while addition of more H₂O resulted in more syn product 18b as determined by ¹H NMR.

In order to minimize the amount of AlMe₃ employed while keeping the ratio constant at 5:1, it was revealed that a minimum of 3 equiv of AlMe₃ must be used giving a dr of 25:1 (Table 5, entry 6) while dr dropped to 5:1 when only 2 equivalents of AlMe₃ were employed (Table 5, entry 7). These results suggest that the best methylating agent is a mixture of AlMe₃ and Me₂AlOAlMe₂ in 1.5:1 ratio respectively and is superior to both AlMe₃, MeAlO or Me₂AlOAlMe₂ by themselves.

TABLE 5
Cleavage of epoxide 17 employing aluminoxanes

| Entry | AlMe₃ equiv | H₂O equiv | dr (anti:syn) |
|---|---|---|---|
| 1 | 5 | 5 | — |
| 2 | 5 | 3 | 10:1 |
| 3 | 5 | 1 | 20:1 |
| 4 | 4 | 1 | 14:1 |
| 5 | 3 | 1 | 12:1 |
| 6 | 3 | 0.6 | 25:1 |
| 7 | 2 | 0.4 | 5:1 |

In addition, the fresh formation of the aluminoxane at 23° C. followed by aging for at least 1 h was helpful in effecting a consistent dr of 25:1. Conditions in entry 6 were successfully scaled up to 100 g and 18a was conveniently obtained by crystallization from diethyl ether, as proven by ¹H NMR and crystal structure.

The workup to quench/eliminate AlMe₃ was a concern on such large scale. Three different workup procedures were investigated. Precipitation of aluminum as alum employing aqueous sodium sulfate at low temperature was successful, but gave low yields due to trapping of 18a in the alum and clogging of the filtration pad. Alternatively, employing excess aqueous NaOH to form soluble aluminate was successful. However, emulsion formation was always an issue, and the quench required hours for completion. The best workup was transfer of the reaction to a mixture of mechanically stirred aqueous HCl-crushed ice. This quench efficiently removed all aluminum and resulted in no formation of emulsion. Thus, crystallization of 18a was facile from boiling ether, or 18a could even be used without further purification. Dianion formation-sulfenylation of 18a was performed on multidecagram scale employing NaHMDS. On such scale, dianion formation is not complete except at 23° C. with mechanical stirring (300 rpm). Lower rpm or stirring bars do not achieve dianion formation on large scale. It was also observed that [0.2 M] reactions are very viscous and do not complete dianion formation, therefore reaction concentration should not be higher than [0.1 M]. Due to the sensitivity of the dianion to moisture, reaction monitoring must not involve any opening to the atmosphere, even under positive pressure of dry inert gas. Instead, an aliquot is withdrawn with a glass syringe (containing dry THF), emptied in a dry vial, then monitored by TLC. Dianion formation could be monitored on TLC since the dianion quenches on silica to an allylsulfone instead of simply returning starting 18a.

Quenching the dianion with a solution of dry PhSSPh cleanly afforded 19 on 60 g scale. Purification by continuous extraction of biphasic CH₃CN-hexane system efficiently removed all PhSSPh with no silica purification required. 19 was received as a single diastereomer in stark contrast to the case of 7.

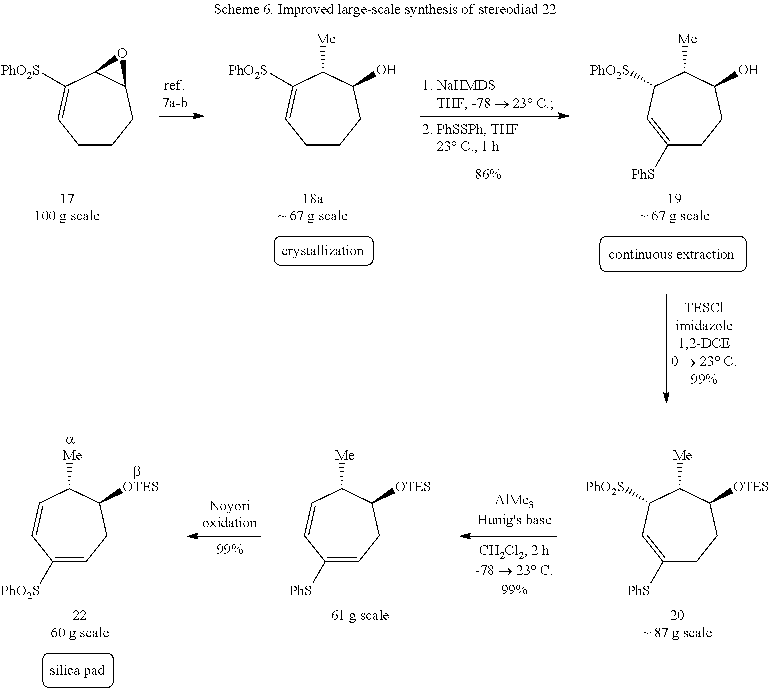

Silylation to 20 employing TESCl and imidazole required only filtration and concentration for workup. Trimethylaluminum-mediated 1,4-elimination of 20 went smoothly at 23° C. after the addition was performed at −78° C. An improved workup procedure was transfer of the reaction contents to a mechanically stirred mixture of aqueous HCl-crushed ice. The TESO-group was not affected by the seemingly harsh conditions, yielding 21 quantitatively.

Noyori oxidation of 21 went smoothly on 60 g scale and the TES 0-group survived once again the presence of peroxide. Short silica pad filtration furnished dienylsulfone 22 as a highly pure clear oil in 99% yield (Scheme 6).

### Jacobsen (Syn) Epoxidation of 22

Epoxidation of 22 to 25 proved to be difficult due to the epoxide being syn to the neighboring methyl group (steric effect). Early attempts to elaborate the syn epoxide 25 via iodination-hydration resulted in the diastereomeric iodohydrin precursor 26 as confirmed by X-ray crystallography (Scheme 7).

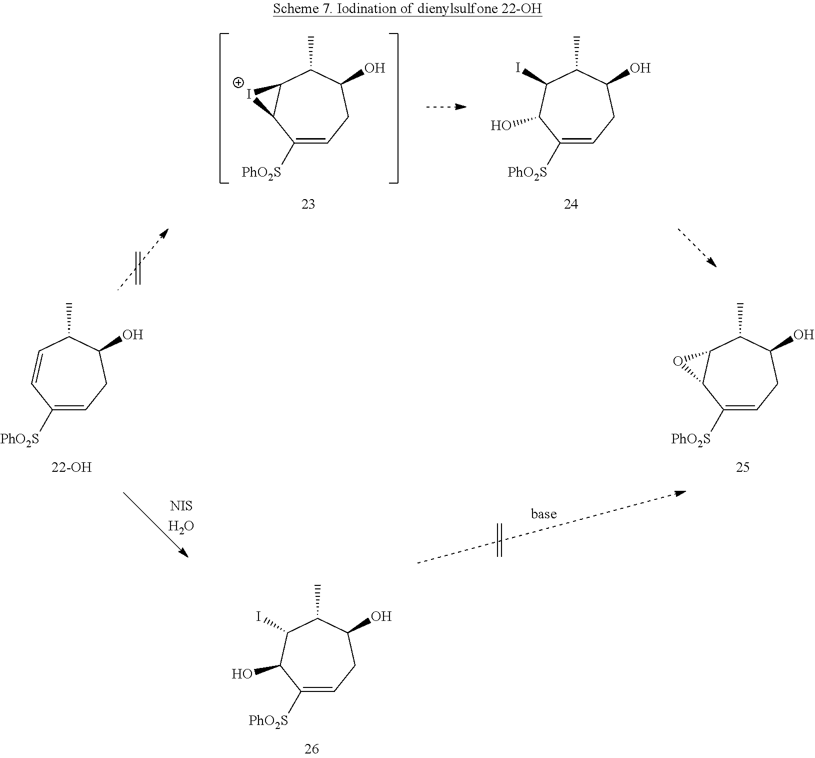

The above result suggested that 22-OH is adopting a conformation where the α-face is unexpectedly the less hindered face. Thus, “direct” epoxidation methods were investigated. Unfortunately, trials involving MCPBA/CH₂Cl₂ or DMDO/acetone proceeded unselectively to give ˜1:1 mixture of diastereomeric epoxides. The Jacobsen epoxidation conditions developed by Torres ((a) J. Org. Chem. 2002, 67, 200, (b) Synthesis 2004, 11, 1895) on unsubstituted dienyl sulfones resulted only in 35% yield due to reaction stalling after 7 days (Table 6, entry 1). Switching to the more reactive oxidant NaOCl employing P₃NO as an axial ligand afforded 25-OTES in 34% under standard Jacobsen conditions (pH 11.4). The reaction had a fast onset followed by stalling after several days with concomitant decomposition of the catalyst and the product 25-OTES. Attempts to add more catalyst did not improve the conversion but rather resulted in product decomposition. When the reaction was performed at 23° C., an immediate exotherm was observed resulting in catalyst decomposition

It was reported that the active oxidant HOCl is slowly released into the organic phase at pH 11.4, and a lower pH might result in chlorinated side products (Zhang, et al. J. Org. Chem. 1991, 56, 2296). However, an increase in yield was observed when pH was lowered to 11.14 (Table 6, entry 3). It was clear that higher concentration of HOCl is required early in the reaction to avoid potential catalyst inhibition. Indeed, yields increased due to consumption of 22 as pH values were lowered, with pH 9.5 being an optimal value. Interestingly, no chlorinated products were detected under these conditions. The pH value was adjusted by mixing freshly prepared cold solutions of 10-13% NaOCl and NaH₂PO₄, followed by adding the mixture as a continuous stream into the cold reaction vessel. However, dropwise addition suffered from the old problem of reaction stalling even at pH 9.5. A viable reaction is dark black and opaque compared to a brown suspension in the cases that fail to go to completion. On multigram scale, the reaction is best performed with mechanical stirring to effect efficient mixing at the solvent interface.

TABLE 6
Large-scale optimization of Jacobsen epoxidation of 22

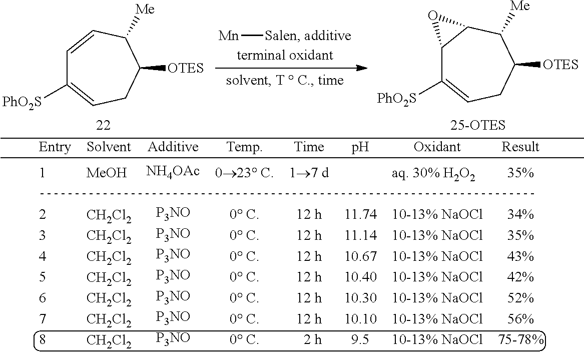

This reagent-matched stereoselective epoxidation was practical on 50 g scale and is considered the lowest pH Jacobsen epoxidation reported to date. Another large-scale problem was the sensitivity of epoxide 25-OTES to silica gel, rendering its purification a difficult task. Moreover, the Manganese (Mn) residues streaking through silica complicated the issue. It was thus desired to effect precipitation of the Mn-salen catalyst while avoiding chromatography. After extensive investigation, it was discovered that adding 300% v/v hexanes to the reaction mixture, and stirring at 0° C. for 1 h quantitatively precipitated the Mn-salen catalyst.

For multigram scale reactions, the mixture was stored at 23° C. for 12 h then was filtered on a celite pad resulting in the complete recovery of the catalyst. If necessary, 0.5 equivalent of 10-13% aqueous NaOCl could be added at 23° C. to effect an exotherm that will destroy the residual catalyst, followed by filtration through celite. Concentration of the filtrate resulted in yields of 75-78% in a highly pure state as judged by ¹H NMR.

### Synthesis of Stereotetrad 2

Similar to epoxide 13, epoxide 25-OTES underwent Lawton S_{N}2′ addition of 3,5-dimethylpyrazole (3,5-DMP) at 50° C. Purification by silica pad filtration is a “must” at this stage due to the sensitivity of the next reaction to the purity of 27. Optimization of syn-directed methylation of 27 is summarized (Table 7), highlighting the importance of the order, temperature, and mode of addition.

Subsequent silylation with TBSOTf, followed by regioselective desilylation of the TESO-group employing CSA afforded 2 as a single diastereomer. Silica pad filtration furnished 2 on 4.5 g scale (Scheme 8).

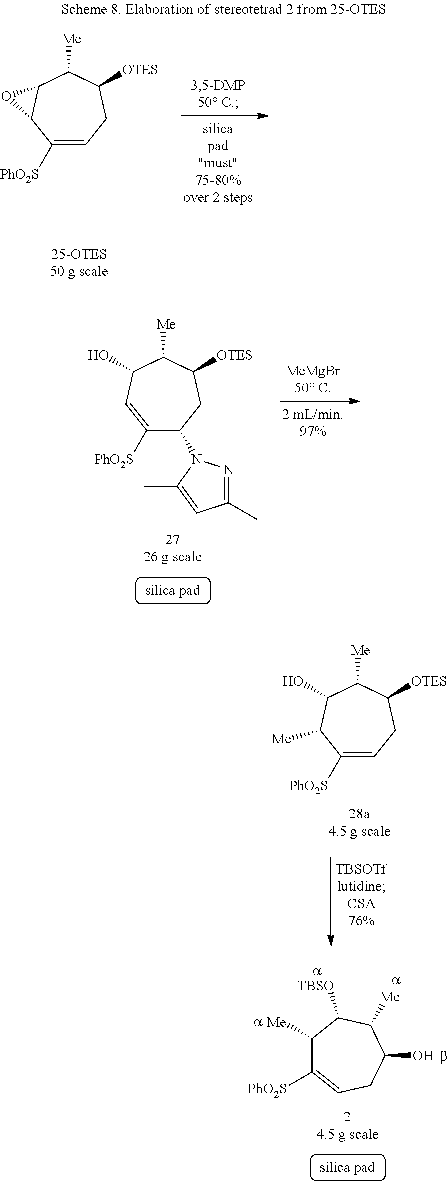

### Syn-Directed Methylation of 27.

Addition of MeMgBr slowly to 27 at 23° C. proceeded without completion and without side product 28b (Table 7, entry 1). Adding excess reagent and/or warming the reaction resulted in increased formation of unwanted 28b. In contrast, rapidly adding MeMgBr improved the ratio of 28a to 27 along with side product 28b (Table 7, entry 2). A one shot addition on a small scale was similar to entry 1, except some 28b was detected. It was clear that reaction onset is accompanied with an exothermic phase, that if exceeded results in the undesired formation of 28b. Thus, a series of experiments were conducted adding MeMgBr to warm 27. At 40° C., different addition rates gave similar results to the fast addition at 25° C. indicating that the reaction was not warm enough to effect consumption of 27. At 60° C., 28a was received in approximately 80%. Only 27 was observed upon dropwise addition of MeMgBr at 50° C. (Table 7, entry 7, 8). It was even practical to add MeMgBr at rates of 1-2 mL/min on scales up to 10 g of 27 affording 28a in 97% yield. No purification was required since ¹H NMR was completely pure.

TABLE 7
Large-scale optimization of syn-directed methylation of 27

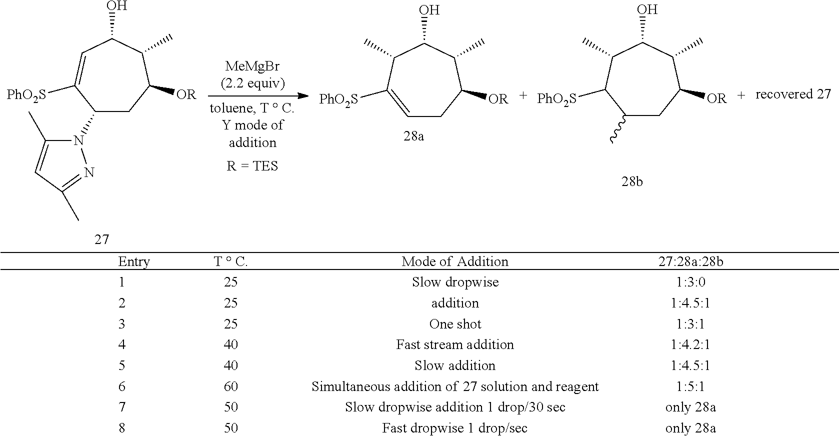

Similarly, ent-17 was transformed to ent-22 over five steps while avoiding chromatography. Conversion of ent-22 to ent-2 was successful over five steps while avoiding chromatography. The entire operation/sequence requires only two silica pad filtrations and uneventfully provides multigram quantities of ent-2 bearing all stereogenic centers for elaboration of C28-C33 of Aplyronine A and analogs (Scheme 9).

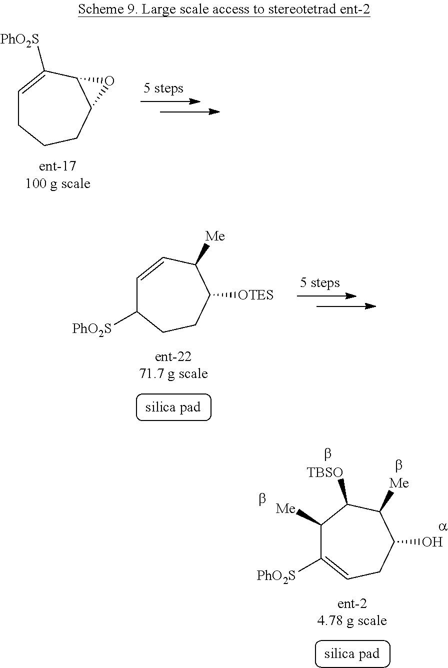

### Ozonolytic Cleavage

Ozonolytic cleavage of vinylsulfone stereotetrads 2 and 16 proved challenging since they are sterically hindered electron-deficient olefins. Ozonolysis in ethyl acetate, dichloromethane, or even methanol did not proceed below −40° C. Although desired lactones such as 27 were obtained, yields were always subject to variation on large scales since high temperature ozonolysis (>−40° C. in this case) is often accompanied with decomposition pathways. Low pH values were observed in CH₂Cl₂ (pH 1-2), ethyl acetate, and even methanol (pH 3-4) presumably due to the oxidation of these solvents by ozone to HCl or carboxylic acids. Addition of bases such as NaHCO₃, pyridine or triethyl amine was beneficial with NaHCO₃ being the most attractive since it does not react with ozone. Large amounts of NaHCO₃ (10 equiv) were required for multigram scale ozonolysis rendering stirring inefficient. In non-trapping solvents, carbonyl oxide 29 undergoes lactone formation to 30 leaving the unprotected carbonyl oxide moiety undergo dimerization to stable tetroxanes such as 31. In contrast, ozonolysis in acetone not only minimized solvent oxidation by ozone but also offered a trapping solvent that protected the carbonyl oxide of 29 as a stable secondary ozonide 32 until the completion of ozonolysis. Acetone must be freshly distilled from CaSO₄, and all starting materials must be azeotroped from toluene before reaction. Late addition of NaHCO₃ and reduction with Me₂S followed by treatment with ^{t}BuNH₂.BH₃ afforded lactones such as 33 in yields up to 80% on multigram scale (Scheme 10).

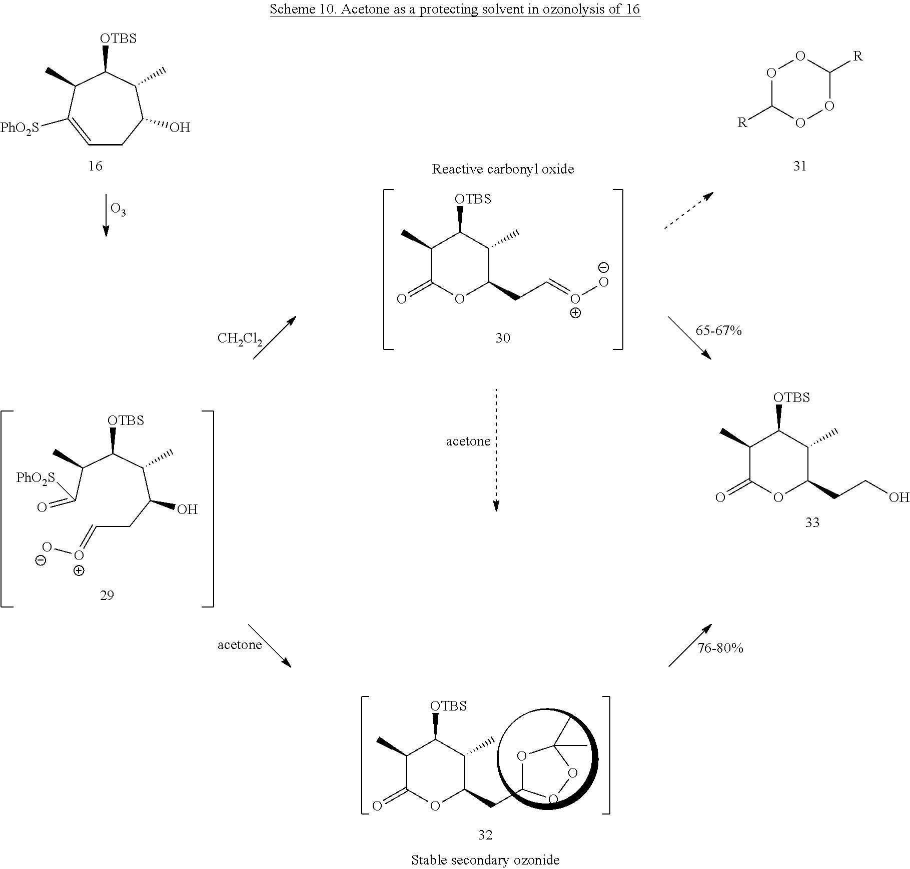

Another protocol for ozonolysis was investigated with catalytic pyridine in order to evade the excessive utilization of solid inorganic salts that minimizes the efficiency of stirring on large scales. While ozone cleaved 16 to 34 pyridine was oxidized to N-oxide 35 that added to 34 to form 36.

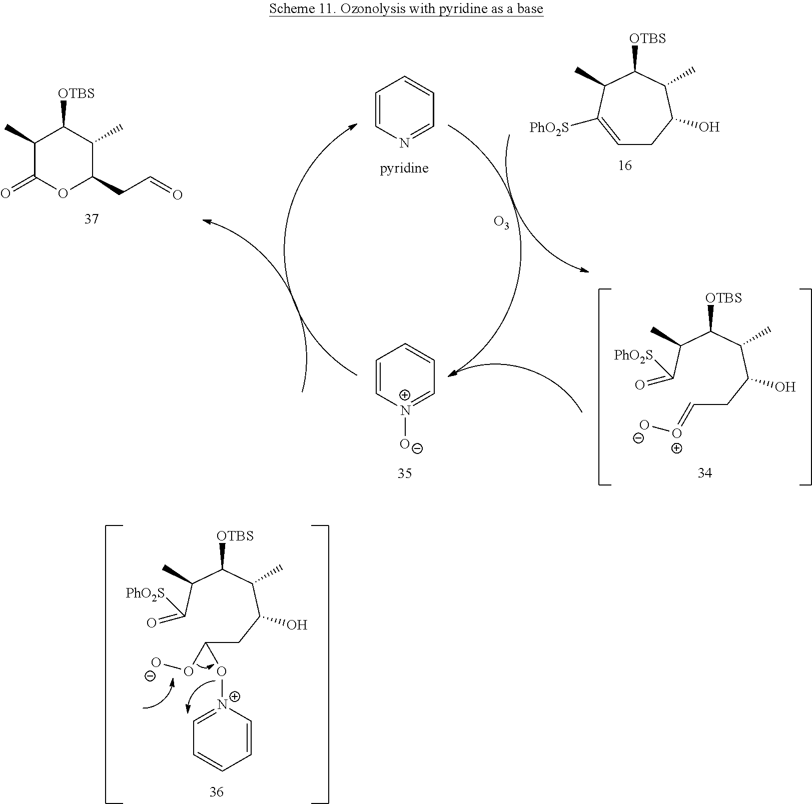

The unstable adduct 36 is proposed to collapse into molecular oxygen, pyridine, and aldehyde 37. Ozonolysis under these conditions afforded aldehyde 37 in 76% yield on 1 gram scale. The pH was basic throughout the reaction course at −30° C. Although the mechanism suggests catalytic involvement of pyridine, it was found that at least 10 equivalents of pyridine are required for a successful reaction (Scheme 11).

Under the optimized conditions, 16 and 2 could be efficiently cleaved by ozonolysis to elaborate the extremely valuable lactones 33 and 38 in 76% and 65% respectively. The importance of such lactones was clearly demonstrated in the synthesis of C6-C10 (39) and C21-C27 (40) of Aplyronine A (Scheme 12).

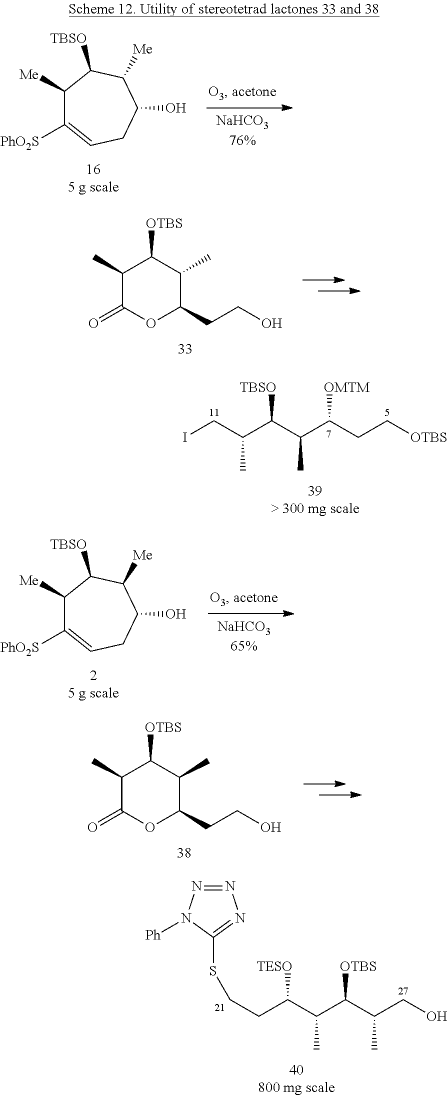

The present invention provides a practical and optimized large-scale access to syn- and anti-cycloheptadienylsulfones 10 and 22 via improved procedures from a common epoxide precursor. It is also provided the first optimized and large scale process of accessing stereotetrads 2 and 16 from 22 and 10 respectively while avoiding chromatography. These valuable stereotetrads underwent ozonolytic cleavage in acetone to yield lactones 33 and 38 on multigram scales. All details of issues related to large scales were addressed and described for both processes for each stereotetrad. The large scale and facilitated access to lactones 33 and 38 was applied towards the elaboration of segments C5-C10 and C21-C27 of aplyronine A and analogs.

The term “reacting” is meant to refer to the bringing together of the indicated reagents in such a way as to allow their molecular interaction and chemical transformation according to the thermodynamics and kinetics of the chemical system. Reacting can be facilitated, particularly for solid reagents, by using an appropriate solvent or mixture of solvents in which at least one of the reagents is at least partially soluble. Reacting is typically carried out for a suitable time and under conditions suitable to bring about the desired chemical transformation.

As used herein, the terms “stereodiad” (or “cycloheptadienylsulfones”) and “stereotetrad” refer to compounds having specific structure features as demonstrated herein, for example, stereotetrads 2 and 16, or stereodiads 10 and 22 (or cycloheptadienylsulfones 10 and 22).

The processes described herein may include other suitable starting materials through the synthetic routes set forth above. In some embodiments, the processes described herein may also include additional steps before or after the steps described above, to add or remove suitable protecting groups. In addition, various synthetic steps may be performed in an alternate sequence or order to give the desired compounds.

This present invention utilizes the chemical properties of vinylsulfones as a highly versatile chemical moiety. Cheap and commercially available cyclic ketones are efficiently transformed into fully functionalized complex polyketides with high overall yields and in pure stereoselective fashion. The cleanliness of transformations allows for industrial scale production of these valuable intermediates without any extensive purification especially the expensive flash column chromatography. This ability to easily scale up the key intermediates is crucial to bringing aplyronine A to clinical trials. In addition to the synthetic power of vinylsulfones, the UV-active sulfone functionality allows for facile monitoring of reactions' progress thus enabling smooth process production.

Further, the introduction of sulfone group in almost all intermediates enables easy purification of large scales via crystallization, which is one of the most industrially preferred techniques for purification of large scale reactions.

The processes of the present invention are economically beneficial. For example, the key intermediate (cycloheptadienylsulfone) was synthesized on a kg scale and the cost analysis revealed that this synthesis costs only 700.

The present invention provides processes for preparation of all the fragments of the ApA molecule on a large scale as described herein. The final step towards ApA involves coupling of those the fragments to afford the final product ApA. All couplings are an assortment of known efficient olefin coupling reactions (e.g. HWE, Julia-Kocienski, Masamune)

The following examples are presented in order to more fully illustrate the preferred embodiments of the invention. They should in no way be construed, however, as limiting the broad scope of the invention.

### EXAMPLES

### General Procedures

All reactions were performed in oven-dried or flame-dried round-bottom flasks. The flasks were fitted with rubber septa and reactions were conducted under a positive pressure of argon. Gas-tight syringes with stainless steel needles or cannulae were used to transfer air- and moisture-sensitive liquids. Flash column chromatography was performed as described by Still et al. using granular silica gel (60-Å pore size, 40-63 μm, 4-6% H₂O content, Zeochem) (Still, et al. J. Org. Chem. 1978, 43, 2923). Analytical thin layer chromatography (TLC) was performed using glass plates pre-coated with 0.25 mm 230-400 mesh silica gel impregnated with a fluorescent indicator (254 nm). TLC plates were visualized by exposure to short wave ultraviolet light (254 nm) and a solution of p-Anisaldehyde stain (PAA), Potassium permanganate (KMnO₄), ceric ammonium molybdate (CAM), or vanillin stain, followed by heating on a hot plate (˜250° C.). Organic solutions were concentrated at 29-30° C. on rotary evaporators capable of achieving a minimum pressure of ˜2 torr.

### Materials

Commercial reagents and solvents were used as received with the following exceptions: dichloromethane, benzene and toluene were distilled over calcium hydride, acetonitrile was pre-dried over calcium hydride then was distilled over calcium hydride, acetone was distilled over calcium sulfate, tetrahydrofuran was distilled from sodium benzophenone ketyl. Pyridine, triethylamine, and Hünig's base were dried over potassium hydroxide pellets for at least 48 hours before use.

### Instrumentation

Proton nuclear magnetic resonance (¹H NMR) spectra are reported in parts per million on the δ scale, and are referenced from the residual proton in the NMR solvent (CDCl₃: δ 7.26 (CHCl₃), CD₂Cl₂: δ 5.32 (CHDCl₂), DMSO-d₆: δ 2.50 (DMSO-d₅)). Data are reported as follows: chemical shift [multiplicity (s=singlet, d=doublet, t=triplet, sp=septet, m=multiplet), coupling constant(s) in Hertz, integration, assignment]. Carbon-13 nuclear magnetic resonance (¹³C NMR) spectra were reported in parts per million on the δ scale, and are referenced from the carbon resonances of the solvent (CDCl₃: δ 77.00, CD₂Cl₂: δ 54.00, DMSO-d₆: δ 39.51). Peak assignments of intermediates are based on analyses of gradient COSY experiments and chemical shifts of individual protons. High resolution mass spectra (HRMS) were recorded on using either an electrospray (ESI) or direct analysis in real time (DART) ionization source.

### Example 1: Synthesis of Allylsulfone 6-AS

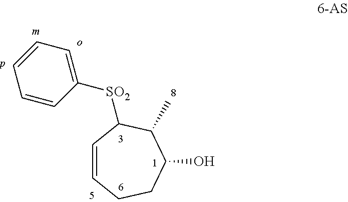 [SMILES: C[C@H]1C(S(=O)(=O)c2ccccc2)C=CCC[C@H]1O]

A 15 mL dry RB flask was charged with dry dienyl alcohol 5 (50 mg, 0.2 mmol, 1 equiv) dissolved in freshly distilled dry THF (2.0 mL). The mixture was stirred at 25° C. for 10 minutes then was cooled under Ar in an acetone-dry ice bath for 30 minutes. Grignard reagent 1.4 M MeMgBr in THF-toluene (75:25) (314 uL, 0.44 mmol, 2.2 equiv) was added at −78° C. dropwise, then the dry-ice bath was removed. The reaction was monitored by TLC (50% ethyl acetate in hexanes; PAA stain) until starting material disappeared while a higher Rf red spot appeared. The reaction mixture was diluted with ether (6.0 mL) then transferred via cannula to cold aqueous 5% HCl (8 mL) while stirring. The aqueous phase was discarded, and the organic layer was washed with brine and dried over Na₂SO₄ for 30 minutes. Removal of solvents via rotary evaporation gave a crude yellow oil. Purification via flash column chromatography (80% ether in hexanes) afforded allyl sulfone 6-AS as a colorless oil (51 mg, 96%). TLC (50% ethyl acetate in hexanes; PAA stain) Rf 0.65; ¹H NMR (CDCl₃, 300 MHz) δ 7.90 (dt, J=1.5, 7.2 Hz, 2H, SO₂Ph o-protons), 7.65 (tt, J=2.4, 7.2 Hz, 1H, SO₂Ph p-proton), 7.55 (tt, J=0.9, 7.2 Hz, 2H, SO₂Ph m-protons), 6.18 (ddd, J=3.6, 9.0, 12.0 Hz, 1H), C₄H, 5.23 (dddd, J=0.6, 3.0, 8.1, 11.7 Hz, 1H, C₅H), 4.64 (dt, J=3.6, 10.2 Hz, 1H, C₃H), 3.67 (dd, J=3.3, 8.1 Hz, 1H, C₁H), 2.94 (m, 1H, C₂H), 2.50 (m, 1H, C₆H), 2.24 (m, 1H, C₆H), 1.77 (m, 1H, C₇H), 1.72 (t, J=4.2 Hz, 1H, C₇H), 1.68 (dd, J=3.3, 12.0 Hz, 1H, hydroxyl proton), 1.04 (d, J=6.9 Hz, 3H, C₈H₃, methyl protons); ¹³C NMR (CDCl₃, 75 MHz) δ 140.1, 139.2, 133.6, 129.0, 128.7, 119.5, 71.4, 68.9, 35.5, 28.4, 24.1, 12.2; LRMS (ESI) calculated for [C₁₄H₁₈O₃S+Na]+289. found, 289; HRMS (ESI) calculated for [C₁₄H₁₈O₃S+Na]+289.0874. found, 289.0872.

### Example 2: Synthesis of Sulfenyl Vinylsulfones 6

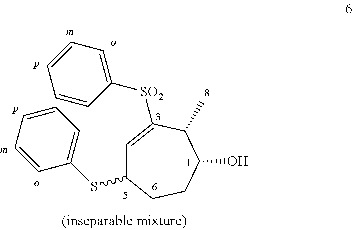 [SMILES: C[C@H]1C(S(=O)(=O)c2ccccc2)=CC(Sc2ccccc2)CC[C@H]1O]

A 3 L three-necked flask was fitted with a mechanical stirrer shaft and two rubber septa then was flame-dried under a stream of dry N₂ then was flushed with dry Ar. Hydroxydienylsulfone 5 (70.0 g, 280 mmol, 1 equiv) was dissolved in dry THF (200 mL) then was transferred to reaction flask via cannula. The flask containing 5 was further washed with dry THF (2×50 mL), and the washings were transferred to reaction flask via cannula. Stirring was maintained at 300 rpm then the reaction was cooled to −78° C. in acetone-dry ice bath for 30 minutes. MeMgBr 1.4 M in THF/toluene (75:25) (440 mL, 616 mmol, 2.20 equiv) was transferred from a flame-dried graduated cylinder under a stream of dry N₂ to reaction flask at −78° C. over 15 minutes. The cylinder was further washed with dry THF (50 mL), and the washings were transferred to reaction flask via cannula. The acetone-dry ice bath was removed, and the reaction was stirred for 45 minutes after which TLC showed disappearance of starting material 5.

Diphenyl disulfide (86.63 g, 392.0 mmol, 1.400 equiv) dissolved in dry THF (100 mL) was added to reaction mixture via syringe (20 mL at a time) at 25° C. over 20 minutes. The flask containing PhSSPh was further washed with dry THF, and the washings were added to the stirring reaction mixture as described above. A slight exotherm was observed after quenching the in-situ formed anion with PhSSPh (25° C. rises to 35° C.) indicating a successful reaction. Stirring was maintained for 1 h at 25° C. and the reaction was monitored for completion by TLC (50% ethyl acetate in hexanes, PAA stain). Ice-chips (1-2 lbs) were added slowly at 25° C. in order to effect a controlled quench while avoiding possible exotherm, when the reaction transformed into a tan-slush. Stirring was maintained for 30 minutes then aqueous 5% HCl (1.02 L, 1.40 Mole, 5.00 equiv) was slowly added over 10 minutes, and the reaction was further stirred for another 30 minutes. Stirring was stopped, phases were allowed to separate (upper organic layer is dark brown while lower aqueous phase is pale yellow), and aqueous phase was neutralized and discarded. The organic phase was then washed with brine (2×1 L) and the aqueous phases were discarded. Organic solvents were removed via rotary evaporation to give 6 as a crude dark brown oil (containing PhSSPh and PhSH which were removed by continuous extraction). It is noted that complete removal of all organic solvents especially THF is crucial for a successful purification with CH₃CN-hexanes continuous extraction.

### Example 3: Continuous Extraction of 6

The procedure for continuous extraction (although a difference in polarity exists between 20 and its diastereomer 7a/7b, the continuous extraction employing CH₃CN-hexanes was successful for both compounds) of 19 was employed, and yielded 6 as an amber colored oil (92 g, 94%). ¹H NMR (CDCl₃, 300 MHz) δ 7.79 (d, J=8.1 Hz, “2.8” H), 7.61 (t, J=7.2 Hz, 1H), 7.46-7.29 (ap m, “7.2” H), 7.20 (d, J=4.2 Hz, 1H), 4.11 (q, J=3.9 Hz, “0.36” H), 3.98 (m, 1H), 3.59 (m, 1H), 2.93 (m, “0.37” H), 3.86 (m, 1H), 2.33 (q, J=11.1 Hz, “0.4” H), 2.13-2.01 (ap m, “2.86” H), 1.88 (ap m, “2.8” H), 1.80-1.66 (ap m, “1.8” H), 1.07 (d, J=7.2 Hz, “1” H), 0.98 (d, J=7.2 Hz, 3H); ¹³C NMR (CDCl₃, 75 MHz) δ 146.0, 144.5, 143.9, 140.3, 138.8, 138.3, 133.6, 133.5, 133.3, 132.7, 132.6, 131.8, 129.1, 128.2, 128.0, 127.9, 127.5, 77.2, 71.8, 69.8, 69.1, 46.0, 45.4, 39.3, 39.1, 31.8, 31.2, 28.9, 28.0, 18.7, 13.5, 10.8; HRMS (EI) calculated for C₂₀H₂₂O₃S₂ (M⁺) 374.1010. found 374.1005.

### Example 4: Synthesis of Allylsulfone 7a and 7b

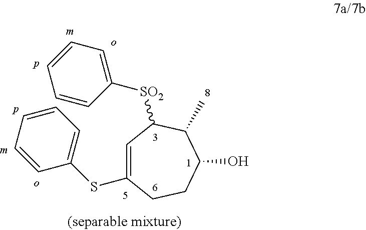 [SMILES: C[C@H]1C(S(=O)(=O)c2ccccc2)C=C(Sc2ccccc2)CC[C@H]1O]

The crude CH₃CN solution [1M] of 6 (67.00 g, 251.9 mmol, 1 equiv) obtained from the continuous extraction above was treated with DBU (1.93 mL, 12.6 mmol, 0.05 equiv) at 25° C. After 15 minutes, the reaction was judged complete by TLC (30% ethyl acetate in hexanes). After solvent removal via rotary evaporation, the resulting brown oil was dissolved in DCE then washed with brine and 5% aqueous HCl (10:1). The organic layer was washed again with brine then dried over Na₂SO₄ for 6 hours. The suspension was filtered over (1″) celite pad, and the filtrate was carried directly to the next step (TESO-protection). Purification of an analytical sample via flash column chromatography (30% ethyl acetate in hexanes) afforded 7a:7b as a diastereomeric mixture (dr ˜2.6:1); TLC (30% ethyl acetate in hexanes) R_{f} 0.27, 0.13.

7a (minor): ¹H NMR (CDCl₃, 400 MHz) δ 7.70 (d, J=7.2 Hz, 2H, SO₂Ph o-protons), 7.65 (t, J=7.6 Hz, 1H, SO₂Ph p-proton), 7.51 (t, J=7.6 Hz, 2H, SO₂Ph m-protons), 7.34 (m, 3H, SPh o, p-protons), 7.32 (m, 2H, SPh m-protons), 5.35 (d, J=6.0 Hz, 1H, C₄H, vinylsulfide proton), 3.73 (dt, J=3.6, 11.2 Hz, 1H, C₃H), 3.69 (d, J=6.4 Hz, 1H, C₁H), 2.88 (m, 1H, C₂H), 2.16 (ap-m, 3H, C₆H+hydroxyl proton), 1.67 (m, 1H, C₇H), 1.52 (m, 1H, C₇H), 1.00 (d, J=6.8 Hz, 3H, C₇H, methyl protons); ¹³C NMR (CDCl₃, 100 MHz) δ 141.7, 138.5, 134.0, 133.7, 131.4, 129.4, 129.2, 128.8, 128.5, 115.7, 76.4, 65.7, 35.1, 29.8, 27.7, 7.8; HRMS (EI) calculated for C₂₀H₂₂O₃S₂ (M⁺) 374.1010. found 374.1006.

7b (major): ¹H NMR (CDCl₃, 400 MHz) δ 7.81 (d, J=7.2 Hz, 2H, SO₂Ph o-protons), 7.62 (t, J=7.2 Hz, 1H, SO₂Ph p-proton), 7.52 (t, J=7.6 Hz, 2H, SO₂Ph m-protons), 7.36-7.26 (m, 5H, SO₂Ph o, m, p-protons), 4.74 (d, J=8.4 Hz, 1H, C₄H, vinylsulfide proton), 4.63 (m, 1H, C₃H), 3.62 (dd, J=3.6, 8.8 Hz, 1H, C₁H), 2.98 (m, 2H, C₂H+hydroxyl proton), 2.18 (m, 2H, C₆H₂), 1.73 (m, 2H, C₇H₂), 1.02 (d, J=6.8 Hz, 3H, C₇H, methyl protons); ¹³C NMR (CDCl₃, 100 MHz) δ 148.3, 139.4, 133.9, 133.5, 131.8, 129.3, 129.0, 128.6, 128.4, 112.4, 71.1, 69.0, 35.4, 30.0, 28.4, 12.2; HRMS (ESI) calculated for C₂₀H₂₂NaO₃S₂ (M⁺) 397.0908. found 397.0906.

Purification of an analytical sample of diastereomeric mixture 7a/7b (30% ethyl acetate in hexanes) afforded 7a as a clear oil (Relative stereochemistry was inferred from the chemical correlation between ¹H NMR and X-ray crystal structure of the acetate derivative 12.). It was observed that 7a cyclizes photolytically upon storage in light (see the characterization of cyclized 11 below). ¹H NMR (CDCl₃, 300 MHz) δ 7.70 (dd, J=1.5, 7.2 Hz, 2H), 7.64 (dt, J=1.2, 6.0 Hz, 1H), 7.30 (d, J=7.8 Hz, 2H), 7.40 (d, J=2.4 Hz, 1H), 7.38 (J=1.2, 3.9 Hz, 2H), 7.30 (m, 2H), 5.35 (d, J=6.0 Hz, 1H), 3.73 (dt, J=3.3, 11.1 Hz, 1H), 3.67 (d, J=0.6, 6.0 Hz, 1H), 2.88 (m, 1H), 2.16 (m, 2H), 1.83 (bs, 1H), 1.70 (m, 1H), 1.51 (m, 1H), 1.0 (d, J=6.9 Hz, 3H, C₇H₃); ¹³C NMR (CDCl₃, 75 MHz) δ 141.7, 138.5, 134.0, 133.7, 131.4, 129.5, 129.2, 128.8, 128.5, 115.7, 76.4, 65.8, 35.2, 29.8, 27.8, 7.8; LRMS (ESI) calculated for [C₂₀H₂₂O₃S₂+Na]⁺ 397. found 397, HRMS (ESI) calculated for [C₂₀H₂₂O₃S₂+Na]⁺ 397.0908. found 397.0907.

### Example 5: Synthesis and Characterization of Cyclized 11

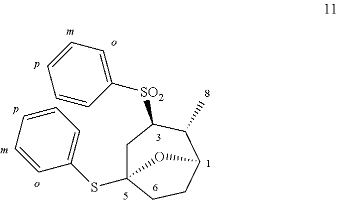 [SMILES: C[C@H]1[C@H](S(=O)(=O)c2ccccc2)C[C@]2(Sc3ccccc3)CC[C@H]1O2]

Allylsulfone 7a (50.0 mg, 0.133 mmol, 1 equiv) was exposed neat to room light and was tested for complete cyclization by TLC (30% ethyl acetate in hexanes). After two weeks, TLC showed a single spot that was purified via flash column chromatography (10% ethyl acetate in hexanes) to afford oxolane 11 as a colorless oil (44.5 mg, 89%); TLC (30% ethyl acetate in hexanes) R_{f} 0.8; ¹H NMR (CDCl₃, 300 MHz) δ 7.79 (dt, J=2.4, 6.9 Hz, 2H, SO₂Ph o-protons), 7.64 (tt, J=0.9, 7.8, Hz, 1H, SO₂Ph p-proton), 7.54 (m, 4H, SO₂Ph m-protons+SPh o-protons), 7.29 (m, 3H, SPh m, p-protons), 4.20 (d, J=6.3, 1H, C₁H), 2.89 (ddd, J=4.2, 5.4, 9.0 Hz, 1H, C₃H), 2.27 (ddd, J=4.2, 8.1, 12.9 Hz, 1H, C₄H), 2.11 (m, 5H, C₂H+C₄H+C₆H₂+C₇H), 1.86 (ddt, J=1.2, 6.0, 12.3 Hz, 1H, C₇H), 1.14 (d, J=7.2 Hz, 3H, C₇H, methyl protons); ¹³C NMR (CDCl₃, 75 MHz) δ 138.2, 134.8, 133.6, 131.6, 129.2, 128.7, 128.4, 128.3, 88.2, 80.8, 63.2, 35.5, 33.7, 32.5, 29.6, 22.5; LRMS (CI/EI) calculated for C₂₀H₂₂O₃S₂ 374. found 374; HRMS (CI/EI) calculated for C₂₀H₂₂O₃S₂ 374.1010. found 374.1012.

### Example 6: Isolation of Allyl Sulfone 7b

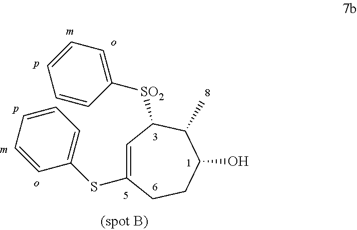 [SMILES: C[C@@H]1[C@H](O)CCC(Sc2ccccc2)=C[C@@H]1S(=O)(=O)c1ccccc1]

Purification of an analytical sample of diastereomeric mixture 7a/7b (30% ethyl acetate in hexanes) afforded 7b (Relative stereochemistry was confirmed by X-ray crystal structure of the acetate derivative. See crystal structure data.) as a clear oil. ¹H NMR (CDCl₃, 300 MHz) δ 7.70 (dd, J=1.2, 7.2 Hz, 2H, SO₂Ph o-protons), 7.64 (dt, J=1.2, 7.5 Hz, 1H, SO₂Ph p-proton), 7.51 (t, J=7.5 Hz, 2H, SO₂Ph m-protons), 7.39 (d, J=2.7 Hz, 1H, SPh p-proton), 7.38 (dt, J=0.9, 3.3 Hz, 2H, SPh o-protons), 7.31 (m, 2H, SPh m-protons), 5.35 (d, J=6.0 Hz, 1H, C₄H, vinylsulfide proton), 3.73 (dt, J=3.9, 11.1 Hz, 1H, C₃H), 3.68 (d, J=6.0 Hz, 1H, C₁H), 2.88 (dt, J=6.9, 10.8 Hz, 1H, C₂H), 2.16 (dd, J=4.2, 9.9 Hz, 2H, C₆H₂), 2.00 (bs, 1H, hydroxyl proton), 1.68 (m, 1H, C₇H), 1.51 (m, 1H, C₇H), 1.00 (d, J=6.6 Hz, 3H, C₇H, methyl protons); ¹³C NMR (CDCl₃, 75 MHz) δ 141.7, 138.5, 134.0, 133.7, 131.4, 129.4, 129.2, 128.8, 128.5, 115.7, 76.4, 65.8, 35.1, 29.8, 27.8, 7.8; LRMS (ESI) calculated for [C₂₀H₂₂O₃S₂+Na]⁺ 397. found 397, HRMS (ESI) calculated for [C₂₀H₂₂O₃S₂+Na]⁺ 397.0908. found 397.0914.

### Example 7: Synthesis of Silyl Ether 8 from Vinyl Sulfide 7

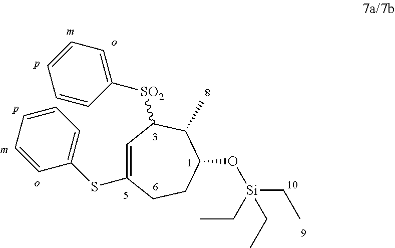 [SMILES: CC[Si](CC)(CC)O[C@@H]1CCC(Sc2ccccc2)=CC(S(=O)(=O)c2ccccc2)[C@@H]1C]

A 3-neck 1 liter flask was flame dried under dry N₂ atmosphere then was charged with a solution of thoroughly dried 7a/7b (65.00 g, 171.9 mmol, 1 equiv) in anhydrous DCE (430 mL) at 25° C. The reaction was cooled to 0° C. then solid imidazole (11.7 g, 172.0 mmol, 1 equiv) was added followed by slow stream addition of TESCl (29.4 mL, 172.0 mmol, 1 equiv) over 10 minutes. After the reaction shows yellowish precipitation of imidazolium hydrochloride, stirring was continued for 1-2 hours at 0 to 25° C. The reaction was checked for completion by TLC (50% ethyl acetate in hexanes; PAA stain) then was filtered over a celite pad (2.5×2.5 in., eluent dichloromethane) under vacuum. The pad was washed with dichloromethane (2×100 mL), and the combined organic phases were concentrated via rotary evaporation to afford crude 8 as an amber oil that did not require further purification. An analytical sample was obtained via flash column chromatography (10% ethyl acetate in hexanes) as a clear yellow oil (97% recovery). TLC (50% ethyl acetate in hexanes; PAA stain) R_{f} 0.9. The crude oil was deemed pure enough by ¹H NMR to carry to the next step, whereas silica pad filtration at this stage is optional.

### Example 8: Synthesis of Dienyl Sulfide 9 from Silyl Ether 8

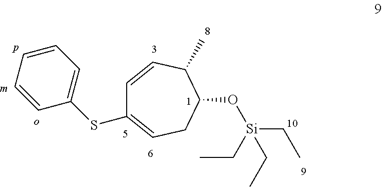 [SMILES: CC[Si](CC)(CC)O[C@@H]1CC=C(Sc2ccccc2)C=C[C@@H]1C]

Crude 8 (35.0 g, 71.7 mmol, 1 equiv) was dissolved in dry DCM (180 mL) then was transferred to a 3-neck 1 L flask under Ar atmosphere. The flask was cooled to −78° C. until the internal temperature became at least −72° C. then iPr₂NEt (31.40 mL, 179.3 mmol, 2.500 equiv) was added via syringe as a slow stream. Trimethylaluminum 25% in hexane (63.00 mL, 157.7 mmol, 2.200 equiv) was added via cannula as a continuous stream over 15 minutes. The acetone/dry ice bath was removed, and the reaction was allowed to warm to 25° C. then stirring was continued for at least 2 hours.

The reaction was checked for completion via TLC (50% ethyl acetate in hexanes; PAA stain) then the reaction contents were transferred via a Teflon tube to a mechanically stirred suspension of 5% aqueous HCl (518 mL, 717 mmol, 10.0 equiv)/crushed ice. The rate of transfer was monitored to avoid vigorous evolution of methane gas. The original reaction flask was washed with an additional (180 mL) of DCM then was transferred to the mechanically stirred biphasic mixture, and stirring was allowed at 25° C. for 1-2 hours. Stirring was stopped, separation of phases was allowed, then the bottom organic layer was siphoned to a new flask via Teflon tube. The aqueous acidic layer was extracted with DCM (90 mL), and the washings were siphoned to the organic mother liquor. The aqueous phase was checked for the presence of 9, then was neutralized and discarded. The combined organic phases were washed with brine (2×250 mL), then dried over Na₂SO₄ for 12 hours. Filtration over a celite pad was followed by concentration of the filtrate via rotary evaporation to afford 9 as a dark brown oil that did not require further purification (24.8 g, 99%) as judged by TLC and ¹H NMR, and was submitted immediately to the Noyori oxidation step (Dienylsulfide 9 does not store well even at −20° C., and decomposes readily at 23° C. over 12 h. Therefore, it must be submitted immediately to the oxidation step.).

### Example 9: Synthesis of Dienyl Sulfone 10 from Dienyl Sulfide 9

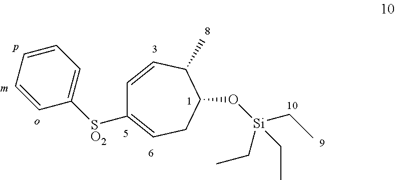 [SMILES: CC[Si](CC)(CC)O[C@@H]1CC=C(S(=O)(=O)c2ccccc2)C=C[C@@H]1C]

[Amines and Halide Salts Interfere with this Reaction].

A 3-neck 2 L flask was fit with an overhead mechanical stirrer, and was placed in an ice-water bath. Dienyl sulfide 9 (24.8 g, 71.7 mmol, 1 equiv) was dissolved in toluene (350 mL), filtered onto a celite pad into the reaction flask (This filtration before the reaction is crucial since Noyori oxidation is halted by the presence of inorganic salts such as chlorides.), and the solution was cooled to 0° C. for 15 minutes. Aqueous 1M Na₂WO₄ (1.4 mL, 1.4 mmol, 0.020 equiv), aqueous 1M PhP(O)(OH)₂ (1.4 mL, 1.4 mmol, 0.020 equiv) and 0.5M oct₃MeNHSO₄ in toluene (2.8 mL, 1.4 mmol, 0.020 equiv) were added and the mixture was stirred at 0° C. for 5 minutes. Cold 30% aqueous H₂O₂ (16.30 mL, 143.4 mmol, 2.000 equiv) was added dropwise then the reaction was allowed to warm up gradually over 2-3 hours without removing the ice bath. The reaction was checked for completion via TLC (50% ethyl acetate in hexanes; PAA stain) then brine (300 mL) was added. The mixture was stirred for 10 minutes then aqueous phase was discarded. The organic layer was dried over Na₂SO₄ then was concentrated via rotary evaporation to afford 10 as a brown oil that was sufficiently pure to carry to the next step. Silica pad filtration is highly recommended in order to remove a black baseline spot, otherwise desilylation of crude product ensues. Silica pad filtration of the above crude oil (2.5×2.5 in; 20% ethyl acetate in hexanes eluent) afforded 10 as a clear yellow oil of high purity as judged by ¹H NMR (26.5 g, 98%). ¹H NMR (CDCl₃, 300 MHz) δ 7.83 (dt, J=1.5, 6.9 Hz, 2H, SO₂Ph o-protons), 7.58 (tt, J=1.5, 7.2 Hz, 1H, SO₂Ph p-proton), 7.45 (tt, J=1.5, 6.9 Hz, 2H, SO₂Ph m-protons), 7.07 (t, J=5.7 Hz, 1H, C₆H, vinylsulfone proton), 6.01 (d, J=11.7 Hz, 1H, C₄H), 5.90 (dd, J=6.3, 11.7 Hz, 1H, C₃H), 4.04 (ddd, J=3.0, 4.8, 8.1 Hz, 1H, C₁H), 2.64 (dd, J=1.5, 4.8 Hz, 1H, C₇H), 2.61 (m, 1H, C₇H), 2.41 (p. J=6.6 Hz, 1H, C₂H), 0.95 (d, J=7.2 Hz, 3H, C₈H₃, methyl protons), 0.91 (t, J=7.8 Hz, 9H, OTES methyl protons), 0.54 (q, J=8.1 Hz, 6H, OTES methylene protons); ¹³C NMR (CDCl₃, 75 MHz) δ 139.8, 139.7, 139.4, 136.7, 133.0, 129.0, 127.6, 118.3, 72.5, 41.6, 36.2, 14.0, 6.7, 4.6.

### Example 10: Synthesis of Epoxide 13 from Dienyl Sulfone 10

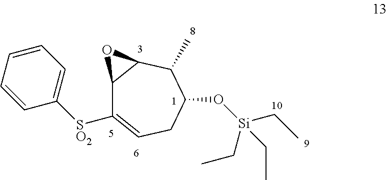 [SMILES: CC[Si](CC)(CC)O[C@@H]1CC=C(S(=O)(=O)c2ccccc2)[C@H]2O[C@H]2[C@@H]1C]

A 3-neck 1 L flask was equipped with an overhead mechanical stirrer, a thermometer, and a rubber septum, then was charged with crude 10 (24.4 g, 64.5 mmol, 1 equiv) dissolved in acetone/DI-H₂O (2:1) (300 mL). Sodium bicarbonate (54.2 g, 645.0 mmol, 10.0 equiv) was added to the solution, and the contents were stirred at 0° C. for 15 minutes. Oxone (39.7 g, 129.0 mmol, 2.000 equiv) was added on four equal doses, with 30 minutes interval between two consecutive additions (It was observed reproducibly that dividing the oxidant dose is more efficient for conversion. One shot addition of oxone often results in reaction stalling, and partial recovery of 10.). After the addition of the last shot of oxone, the reaction was stirred for an additional 1-2 hours while monitoring via TLC determined the reaction conclusion. The reaction contents were filtered on a celite pad, then the pad was washed with ether (300 mL).

The biphasic filtrate was transferred to a separatory funnel where the aqueous layer was separated, and back extracted with ether (if necessary). The combined organic phases were washed with saturated aqueous NaHCO₃, brine then dried over Na₂SO₄ for 12 hours. Solvent removal via rotary evaporation afforded 13 as a crude yellow oil. Filtration through silica pad afforded 13 as a yellow oil (21.9 g, 87%) that was sufficiently pure for the next synthetic step. An analytical sample was obtained via flash column chromatography (30% ethyl acetate in hexanes) as a clear colorless oil (TLC (30% ethyl acetate in hexanes; PAA stain) Rf 0.4; ¹H NMR (CDCl₃, 300 MHz) δ 7.92 (dt, J=1.5, 7.2 Hz, 2H, SO₂Ph o-protons), 7.65 (tt, J=1.8, 7.2 Hz, 1H, SO₂Ph p-proton), 7.56 (tt, J=1.2, 6.9 Hz, 2H, SO₂Ph m-protons), 7.19 (dd, J=3.9, 6.3 Hz, 1H, C₆H, vinylsulfone proton), 3.95 (ddd, J=2.1, 5.2, 7.5 Hz, 1H, C₁H), 3.68 (d, J=4.5 Hz, 1H, C₄H), 3.12 (t, J=4.5 Hz, 1H, C₃H), 2.65 (ddd, J=3.6, 7.8, 18.9 Hz, 1H, C₇H), 2.51 (ddd, J=4.8, 6.0, 19.2 Hz, 1H, C₇H), 2.19 (p, J=6.6 Hz, 1H, C₂H), 1.12 (d, J=7.2 Hz, 3H, C₈H₃, methyl protons), 0.93 (t, J=7.8 Hz, 9H, OTES methyl protons), 0.57 (q, J=7.8 Hz, 6H, OTES methylene protons).

### Example 11: Synthesis of Adduct 14 from Epoxide 13

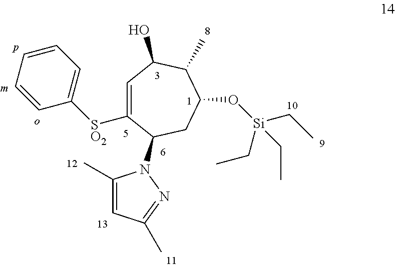 [SMILES: CC[Si](CC)(CC)O[C@@H]1C[C@@H](n2nc(C)cc2C)C(S(=O)(=O)c2ccccc2)=C[C@@H](O)[C@@H]1C]

A 3-neck 1 L flask was fit with an overhead reflux condenser, and two septa. Crude 13 (22.9 g, 58.1 mmol, 1 equiv) was dissolved in dry toluene (150 mL), then was transferred to the reaction flask. Dimethylpyrazole (3,5-DMP) (5.6 g, 58.1 mmol, 1 equiv) was added, then the reaction was heated at 50-60° C. for 1 hour or until TLC shows consumption of 13 (Unnecessary heating after completion result in partial desilylation of 14.). The reaction was cooled to 25° C. then was washed with a mixture of crushed ice (70 ml), brine (70 mL), and 5% aqueous HCl (34.0 mL, 46.5 mmol, 0.800 equiv). The aqueous phase was discarded, then the organic phase was washed with brine, and dried over Na₂SO₄ for 3 hours. Removal of solvents via rotary evaporation afforded 14 as a crude brown oil. Purification via silica pad filtration (2.5×2.5 inch, 30% ethyl acetate in hexanes) afforded 30 as a highly pure crystalline solid (23.3 g, 82%) (High purity at this stage is crucial in order to avoid reaction stalling, or over methylation in the next reaction with MeMgBr.) that was carried to the next synthetic step. ¹H NMR δ 7.64 (d, J=9.0 Hz, 1H, C₄H, vinylsulfone proton), 7.50 (d, J=7.5 Hz, 2H, SO₂Ph o-protons), 7.37 (t, J=7.8 Hz, 1H, SO₂Ph p-proton), 7.24 (t, J=7.2 Hz, 2H, SO₂Ph m-protons), 5.41 (s, 1H, C₁₃H), 5.26 (t, J=3.0 Hz, 1H, C₆H), 4.39 (t, J=3.0 Hz, 1H, C₃H), 4.37 (t, J=3.0 Hz, 1H, C₁H), 4.32 (bs, 1H, hydroxyl proton), 2.34 (m, 1H, C₇H), 2.13 (m, 1H, C₇H), 2.00 (s, 3H, C₁₂H₃, methyl protons), 1.90 (s, 3H, C₁₁H₃, methyl protons), 1.69 (d, J=14.7 Hz, 1H, C₂H), 0.83 (d, J=6.9 Hz, 3H, C₈H₃, methyl protons), 0.73 (t. J=7.5 Hz, 9H, OTES methyl protons), 0.34 (q, J=8.4 Hz, 6H, OTES methylene protons); ¹³C NMR (CDCl₃, 75 MHz) δ 146.7, 145.0, 140.0, 138.7, 138.4, 132.8, 128.4, 126.9, 105.2, 68.4, 64.6, 50.2, 44.6, 36.5, 12.5, 10.3, 9.3, 6.4, 4.4; LRMS (EI) calculated for C₂₅H₃₈N₂O₄SSi (M+) 490. found 490; HRMS (EI) calculated for C₁₄H₁₈O₃S (M+) 490.2322. found 490.2325.

### Example 12: Synthesis of Tetrad 15 from Adduct 14

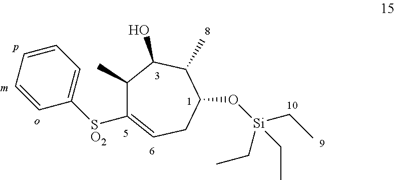 [SMILES: CC[Si](CC)(CC)O[C@@H]1CC=C(S(=O)(=O)c2ccccc2)[C@@H](C)[C@@H](O)[C@@H]1C]

This procedure is sensitive to the degree of purity of 14 (Silica pad filtration is sufficient to obtain a highly pure 14 at this stage.). A 3-neck 100 mL round bottom was fit with a reflux condenser and two rubber septa then was flame dried under a stream of dry N₂. After cooling to 25° C., a solution of 14 (3.0 g, 6.1 mmol, 1 equiv) in dry toluene (30 mL) was transferred to the flask via cannula. The solution was brought to a temperature of 50° C. (internal temperature) then 1.4 MeMgBr in toluene/THF (9.6 mL, 13.5 mmol, 2.20 equiv) was added at a rate of 1 mL/min. such that the internal temperature does not exceed 50° C. The reaction was checked for progress once MeMgBr addition was complete and was found complete.

Quenching at 50° C. with saturated aqueous NH₄Cl (5 mL, careful dropwise addition) was followed by addition of excess saturated aqueous NH₄Cl (50 mL) until all magnesium salts dissolved. The aqueous layer was back extracted with ether (30 mL) then was discarded. The combined organic layers were washed with aqueous 5% HCl, brine then dried over Na₂SO₄ for 4 hours. Removal of solvents via rotary evaporation afforded 15 as a yellow oil (2.3 g, 91%) that was judged highly pure via ¹H NMR. An analytical sample was obtained via flash column chromatography (30% ethyl acetate in hexanes) as a clear oil. TLC (30% ethyl acetate in hexanes; PAA stain) R_{f} 0.32; ¹H NMR (CDCl₃, 300 MHz) δ 7.82 (dt, J=1.5, 6.9 Hz, 2H, SO₂Ph o-protons), 7.56 (tt, J=1.5, 7.2 Hz, 1H, SO₂Ph p-proton), 7.47 (tt, J=1.2, 6.9 Hz, 2H, SO₂Ph m-protons), 7.03 (dd, J=4.8, 8.4 Hz, 1H, C₆H, vinylsulfone proton), 3.93 (d, J=7.2 Hz, 1H, C₃H), 3.36 (dt, J=5.2, 9.9 Hz, 1H, C₁H), 2.75 (dq, J=3.3, 6.9, 2.7 Hz, 1H, C₄H), 2.63 (ddd, J=6.9, 8.4, 15.6 Hz, 1H, C₂H), 2.42 (ddd, J=0.9, 4.8, 15.9 Hz, 1H, C₇H), 1.89 (ddd, J=2.4, 6.6, 12.3 Hz, 2H, C₇H+hydroxyl proton), 1.07 (d, J=7.2 Hz, 3H, C₉H₃, methyl protons), 1.00 (d, J=6.9 Hz, 3H, C₈H₃, methyl protons), 0.92 (t, J=7.8 Hz, 9H, OTES methyl protons), 0.56 (q, J=8.1 Hz, 6H, OTES methylene protons); ¹³C NMR (CDCl₃, 75 MHz) δ 144.0, 139.0, 138.8, 133.0, 128.9, 127.7, 72.3, 70.1, 41.8, 39.4, 34.2, 16.5, 10.7, 6.8, 4.7.

### Example 13: Synthesis of Tetrad 16 from Tetrad 15 (One-Pot)

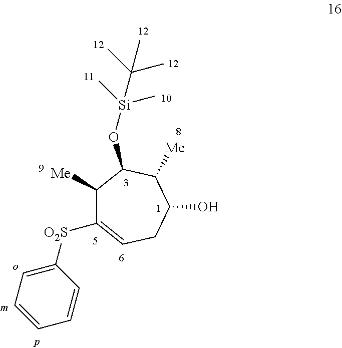 [SMILES: C[C@H]1[C@H](O[Si](C)(C)C(C)(C)C)[C@H](C)C(S(=O)(=O)c2ccccc2)=CC[C@H]1O]

A dry 100 mL round bottom flask was charged with a solution of 15 (10.0 g, 24.4 mmol, 1 equiv) in dry DCM (120 ml), then the solution was cooled to 0° C. After 15 minutes, 2,6-lutidine (3.5 mL, 29.3 mmol, 1.20 equiv) was added dropwise, then TBSOTf (5.7 mL, 24.4 mmol, 1 equiv) was added dropwise via syringe. The reaction was stirred at 0° C. for 30-60 minutes, then MeOH (5.00 mL, 122 mmol, 5.00 equiv) was added dropwise, and stirring was continued for another 30 minutes. The reaction was diluted with 5% aqueous HCl, washed, then the aqueous layer was discarded after neutralization. The organic layer was transferred to another flask, cooled to 0° C., then CSA (2.90 g, 12.2 mmol, 0.50 equiv) was added. After stirring at 0° C. for 2 hours, the reaction was neutralized carefully with sat aqueous NaHCO₃, then washed with saturated aqueous NaHCO₃. The organic layer was then washed with brine, then dried over Na₂SO₄ for 6 hours. Removal of solvents via rotary evaporation afforded crude 16 as a brown oil. Purification via silica pad filtration (eluent 30% EtOAc/hexane) afforded 16 as yellow needle crystals (8.1 g, 81%). [α]²⁵ −61.2 (DCM, c=1).

### Example 14: Synthesis of Diol 16-OH/Ent-16-OH from Tetrad 16/Ent-16

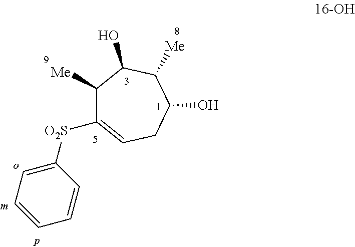 [SMILES: C[C@H]1[C@H](O)[C@H](C)C(S(=O)(=O)c2ccccc2)=CC[C@H]1O]

A 15 mL round bottom flask was charged with a solution of ent-16 (200 mg, 0.49 mmol, 1 equiv) in CH₂Cl₂ (5 mL) at 25° C. The solution was cooled to 0° C. then CSA (116 mg, 0.49 mmol, 1 equiv) was added as a solid or solution in DCM. After five minutes, MeOH (5 drops) was added and stirring was continued at 0 to 25° C. for 2 hours. After checking completion by TLC (50% ethyl acetate in hexanes; PAA stain), saturated aqueous NaHCO₃ was added. The biphasic mixture was transferred to a separatory funnel, shaken then the aqueous phase was discarded. The organic layer was washed with brine, dried over Na₂SO₄ then concentrated via rotary evaporation. The resulting crude oil was purified via flash column chromatography (50% ethyl acetate in hexanes) and afforded the above diol ent-16-OH as a clear yellow oil (132 mg, 92%). TLC (50% ethyl acetate in hexanes; PAA stain) R_{f} 0.1; ¹H NMR (CDCl₃, 300 MHz) δ 7.88 (dt, J=1.8, 6.9 Hz, 2H, SO₂Ph o-protons), 7.62 (tt, J=1.2, 7.2 Hz, 1H, SO₂Ph p-proton), 7.54 (tt, J=1.5, 6.9 Hz, 2H, SO₂Ph m-protons), 7.00 (dd, J=4.5, 8.4 Hz, 1H, C₆H, vinylsulfone proton), 3.98 (d, J=5.7 Hz, 1H, C₁H), 3.57 (d, J=10.5 Hz, 1H, C₃H), 2.86 (dq, J=3.3, 7.2 Hz, 1H, C₄H), 2.80 (ddd, J=6.6, 8.4, 15.3 Hz, 1H, C₇H), 2.50 (ddd, J=1.5, 4.5, 16.2 Hz, 1H, C₇H), 2.28 (bs, D₂O exchangeable, 2H, hydroxyl protons), 2.01 (m, 1H, C₂H), 1.12 (d, J=6.9 Hz, 3H, C₉H₃, methyl protons), 0.94 (d, J=7.2 Hz, 3H, C₈H₃, methyl protons); ¹³C NMR (CDCl₃, 75 MHz) δ 145.0, 138.7, 138.3, 133.4, 129.2, 128.1, 71.8, 70.1, 41.0, 39.4, 33.4, 16.3, 10.5.

### Example 15: Synthesis of Vinylsulfone 18a/Ent-18a

(This procedure was reproducibly repeated on 100 g scale for the furnishing of substantial quantities of ent-18a)

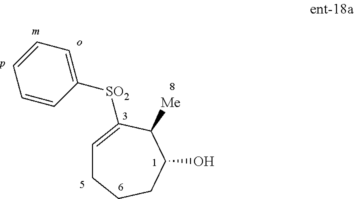 [SMILES: C[C@@H]1C(S(=O)(=O)c2ccccc2)=CCCC[C@H]1O]

A solution of 25% AlMe₃ in hexanes (600.0 mL, 1.20 mol, 3.00 equiv) in dry CH₂Cl₂ (1 L) was stirred with an overhead mechanical stirrer, cooled to −20° C. for 30 minutes then DI-H₂O (4.30 mL, 240.0 mmol, 0.600 equiv) was added dropwise (143 uL/minute) and very carefully over 30 minutes. The dry ice bath was removed and the reaction stirred for 1 h giving a hazy colorless solution. Solid epoxide 17 (100 g, 400 mmol, 1 equiv) was added in portions and the reaction was stirred at 25° C. for 40 minutes. The reaction was checked for completion using TLC (50% ethyl acetate in hexanes; PAA stain) then the reaction contents were transferred via a teflon tube to a mechanically stirred suspension of 5% aqueous HCl (1.30 L, 4.00 mol, 10.0 equiv) and crushed ice. The rate of transfer was monitored to avoid vigorous evolution of methane gas. The original reaction flask was washed with an additional (100 mL) of DCM then was transferred to the mechanically stirred biphasic mixture, and stirring was continued at 25° C. for 1-2 hours. Stirring was stopped, separation of phases was allowed, then the bottom organic layer was siphoned to a new flask via Teflon tube.

The aqueous acidic layer was extracted with DCM (400 mL), and the washings were siphoned to the organic mother liquor. The aqueous phase was checked for the presence of ent-18a, then was neutralized and discarded. The combined organic phases were washed with brine (2×700 mL), then dried over Na₂SO₄ for 12 hours. Filtration over a celite pad was followed by concentration of the filtrate via rotary evaporation to afford ent-18a as an orange oil that did not require further purification as judged by TLC and ¹H NMR. Crystallization from ether gave ent-18a as white prisms (first crop: 66.9 g, 63%, second crop: 9.2 g, 11.9%). Purification of the mother liquor by flash column chromatography (20% ethyl acetate in hexanes) afforded ent-18a as a colorless oil (22.3 g, 21%). MP 74.8-75.4° C.; [α]²⁵ +19.5 (DCM, c=1); TLC (50% ethyl acetate in hexanes; PAA stain) R_{f} 0.26. ¹H NMR (CDCl₃, 300 MHz) δ 7.88 (d, J=8.2 Hz, 2H, SO₂Ph o-protons), 7.58 (m, 3H, SO₂Ph m, p-protons), 7.37 (ddd, J=1.1, 4.1, 9.4 Hz, 1H, C₄H, vinylsulfone proton), 3.82 (br-m, 1H, C₁H), 2.91 (ap. p, J=7.2 Hz, 1H, C₂H), 2.48 (m, 1H, C₅H), 2.27 (tt, J=4.5, 13.0 Hz, 1H, C₅H), 1.98 (m, 1H, C₇H), 1.87 (dt, J=3.1, 12.6 Hz, 1H, C₇H), 1.68 (dt, J=2.0, 12.2 Hz, 1H, C₆H), 1.54 (m, 1H, C₆H), 1.44 (d, J=2.7 Hz, 1H, hydroxyl proton), 0.92 (d, J=7.0 Hz, 3H, C₈H₃, methyl protons); ¹³C NMR (CDCl₃, 75 MHz) δ 144.1, 142.4, 138.8, 133.2, 129.2, 128.4, 69.4, 39.9, 31.4, 27.3, 18.2, 15.6; LRMS (EI) calculated for C₁₄H₁₈O₃S (M+) 266. found 266; HRMS (EI) calculated for C₁₄H₁₈O₃S (M+) 266.0977. found 266.0978.

### Example 16: Synthesis of Sulfenyl Allylsulfone 19/Ent-19

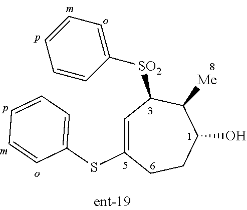

A solution of azeotropically dried alcohol ent-18 (70.00 g, 280.1 mmol, 1 equiv) in THF (634 mL) was cooled to −78° C. for 45 minutes with mechanical stirring under Ar, then 2M NaHMDS in THF (463 mL, 924 mmol, 3.30 equiv) was added fast dropwise over 20 minutes to give a clear yellow solution that eventually turns into deep red clear solution. The dry ice bath was removed and the mixture was stirred at 25° C. for 4 h to give a bright orange suspension. Reaction is checked for complete dianion formation using TLC (50% ethyl acetate in hexanes; PAA stain) (Dianion quenches on TLC to the allyl counterpart and not back to 18a. TLC sampling must be with a syringe having THF since the reaction is viscous. Any moisture introduced to the dianion result to recovered mixture of 18a and its allyl counterpart.).

A solution of PhSSPh (61.62 g, 280.1 mmol, 1 equiv) in THF (280 mL) was added via cannula at 25° C. to the reaction mixture where the orange suspension dissolves signifying successful sulfenylation then a brownish orange suspension reforms immediately signifying dianion precipitation. After stirring for 1 h, reaction was quenched with 5% HCl (700 mL) and diluted with ether (650 mL), and the mixture was stirred for 1 h. After discarding the aqueous phase, a solution of K₂CO₃ (392 g, 2.79 mol, 5.00 equiv) in DI-H₂O (900 mL) was added and the mixture was stirred at 25° C. for another 1 h. The organic phase was separated, washed with brine and dried over Na₂SO₄ for 2 h. Concentration of the organic phase via rotary evaporation afforded ent-19 as a brown oil. Importantly, any residual THF must be removed via rotary evaporation before proceeding to continuous extraction.

### Example 17: Continuous Extraction Purification of 19/Ent-19

The brown oil from above was dissolved in CH₃CN (600 mL) and was transferred to a continuous extraction apparatus that is fit with an overhead Frederick's condenser and a side arm 1000 mL round bottom flask. Hexane (400 mL) was added to give a biphasic system where the lower layer is the CH₃CN layer (clear, brown) and the upper is the hexane layer (clear, colorless). The inner glass tube was filled completely with hexanes and inserted into the extractor while the side arm flask containing hexanes was placed in an oil bath and was heated at 80° C. The extraction was continued for 16 h during which the hexane layer turns yellow and the color is gradually transferred to the side arm flask. After cooling the system to 25° C., the phases were separated and concentrated via rotary evaporation to give highly pure (as checked by ¹H NMR) vinyl sulfide ent-19 as a brown oil (83.4 g, 85%). Crystallization from boiling ether afforded ent-19 as tan crystals (67.6 g, 69%). Repeating the above procedure employing alcohol ent-18a (1.0 g, 4 mmol) followed by purification via flash column chromatography (20% ethyl acetate in hexanes) afforded ent-ent-19 as a yellowish crystalline solid (1.2 g, 86%). MP range 114.4-117.8° C.; [α]²⁵ +39.2 (DCM, c=1); TLC (50% ethyl acetate in hexanes; PAA stain) R_{f} 0.5; ¹H NMR (CDCl₃, 300 MHz) δ 7.60 (dt, J=1.5, 7.2 Hz, 2H, SO₂Ph o-protons), 7.51 (tt, J=2.1, 5.1 Hz, 1H, SO₂Ph p-proton), 7.39 (tt, J=1.5, 7.8 Hz, 2H, SO₂Ph m-protons), 7.26 (m, 3H, SPh o, p-protons), 7.18 (m, 2H, SPh m-protons), 5.22 (d, J=6.0 Hz, 1H, C₄H, vinylsulfide proton), 4.47 (d, J=6.0 Hz, 1H, C₃H), 3.84 (br. m, 1H, C₁H), 2.81 (br-m, 1H, C₂H), 2.70 (dd, J=13.2, 15 Hz, 1H, C₆H), 2.59 (dq, J=7.5, 11.2 Hz, 1H, C₆H), 1.63 (m, 2H, C₇H+hydroxyl proton), 1.50 (dtt, J=2.1, 4.5, 14.7 Hz, 1H, C₇H), 0.95 (d, J=7.2 Hz, 3H, C₈H₃, methyl protons); ¹³C NMR (CDCl₃, 75 MHz) δ 142.7, 138.5, 133.7, 133.4, 131.6, 129.2, 128.9, 128.4, 128.3, 116.6, 72.2, 62.5, 35.3, 26.4, 26.3, 12.7; LRMS (EI) calculated for C₂₀H₂₂O₃S₂ (M+) 374. found 374; HRMS (EI) calculated for C₂₀H₂₂O₃S₂ (M+) 374.1012. found 374.1010.

### Example 18: Synthesis of Silyl Ether 20/Ent-20

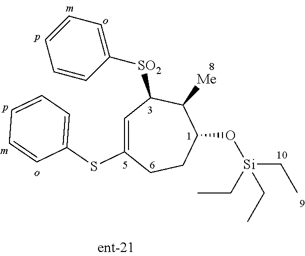

A 3-neck 1 liter flask was flame dried under dry N₂ atmosphere then was charged with a solution of azeotropically dried ent-19 (100.0 g, 267.4 mmol, 1 equiv) in anhydrous 1,2-dichloroethane DCE (660 mL) at 25° C. The reaction was cooled to 0° C. then solid imidazole (20.0 g, 294.1 mmol, 1.10 equiv.) was added followed slow stream addition of TESCl (45.8 mL, 267.4 mmol, 1.0 equiv.) over 15 minutes. After the reaction showed yellowish precipitation of imidazolium hydrochloride, stirring was continued for 1-2 hours at 0 to 25° C. The reaction was checked for completion by TLC (50% ethyl acetate in hexanes; PAA stain) then was filtered over a celite pad under vacuum. The pad was washed with 1,2-dichloroethane DCE (3×100 mL), and the combined organic phases were concentrated via rotary evaporation to afford crude 20 as an amber oil (126.8 g, 98%) that did not require further purification. An analytical sample was obtained via flash column chromatography (10% ethyl acetate in hexanes) as a clear yellow oil. TLC (50% ethyl acetate in hexanes; PAA stain) R_{f} 0.9; ¹H NMR (CDCl₃, 300 MHz) δ 7.74 (tt, J=1.2, 7.2 Hz, 2H, SO₂Ph o-protons), 7.63 (tt, J=1.2, 7.5 Hz, 1H, SO₂Ph p-proton), 7.52 (t, J=7.2 Hz, 2H, SO₂Ph m-protons), 7.36 (m, 5H, SPh o, m, p-protons), 5.55 (d, J=6.0 Hz, 1H, C₄H, vinylsulfide proton), 4.60 (d, J=6.0 Hz, 1H, C₃H), 3.85 (t, J=5.2 Hz, 1H, C₁H), 2.84 (t, J=14.7 Hz, 1H, C₂H), 2.49 (m, 1H, C₆H), 1.75 (m, 1H, C₆H), 1.70 (d, J=13.8 Hz, 1H, C₇H), 1.44 (dt, J=4.8, 14.4 Hz, 1H, C₇H), 1.06 (d, J=7.2 Hz, 3H, C₈H₃, methyl protons), 0.91 (t, J=8.1 Hz, 9H, OTES methyl protons), 0.52 (q, J=8.1 Hz, 6H, OTES methylene protons); ¹³C NMR (CDCl₃, 75 MHz) δ 142.4, 138.8, 133.5, 133.3, 132.1, 129.3, 129.0, 128.5, 128.3, 117.4, 72.8, 62.6, 36.3, 27.5, 26.4, 12.6, 6.8, 4.7.

### Example 19: Synthesis of Dienyl Sulfide 21/Ent-21

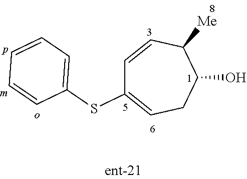 [SMILES: C[C@@H]1C=CC(Sc2ccccc2)=CC[C@H]1O]

Crude 20 (99.1 g, 202.8 mmol, 1 equiv) was dissolved in dry DCM (500 mL) then was transferred to a 3-neck 1 L flask under Ar atmosphere. The flask was cooled to −78° C. until the internal temperature became at least −72° C. then iPr₂NEt (89.0 mL, 507.0 mmol, 2.50 equiv) was added via cannula as a slow stream. Trimethylaluminum 25% in hexane (187.0 mL, 446.4 mmol; 2.30 equiv.) was added via cannula as a continuous stream over 15 minutes. The acetone-dry ice bath was removed, and the reaction was allowed to warm to 25° C. then stirring was continued for at least 2 hours. The reaction was checked for completion via TLC (50% ethyl acetate in hexanes; PAA stain) then the reaction contents were transferred via a teflon tube to a mechanically stirred suspension of 5% aqueous HCl (1.30 L, 1.78 mol, 10.0 equiv) and crushed ice. The rate of transfer was monitored to avoid vigorous evolution of methane gas. The original reaction flask was washed with an additional (100 mL) of DCM then was transferred to the mechanically stirred biphasic mixture, and stirring was allowed at 25° C. for 1-2 hours. Stirring was stopped, separation of phases was allowed, then the bottom organic layer was siphoned to a new flask via Teflon tube.

The aqueous acidic layer was extracted with DCM (400 mL), and the washings were siphoned to the organic mother liquor. The aqueous phase was checked for the presence of 21, then was neutralized and discarded. The combined organic phases were washed with brine (2×700 mL), then dried over Na₂SO₄ for 12 hours. Filtration over a celite pad was followed by concentration of the filtrate via rotary evaporation to afford 21 as a dark brown oil (75.2 g, 98%) that did not require further purification as judged by TLC and ¹H NMR, and was submitted immediately to Noyori oxidation (Dienylsulfide 21 does not store well at −20° C., and decomposes readily at 23° C. over 12 h. It must be submitted immediately to the oxidation step.).

### Example 20: Synthesis of Dienyl Sulfone 22/Ent-22

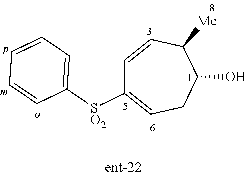 [SMILES: C[C@@H]1C=CC(S(=O)(=O)c2ccccc2)=CC[C@H]1O]

### Small Scale Procedure

Dienyl sulfide 21 (320 mg, 1.38 mmol, 1 equiv) was dissolved in toluene (14 mL) and the solution was cooled to 0° C. for 15 minutes. Aqueous 1M Na₂WO₄ (28 μL, 0.028 mmol, 0.020 equiv), aqueous 1M PhP(O)(OH)₂ (28 μL, 0.028 mmol, 0.02 equiv) and 0.5M oct₃MeNHSO₄ in toluene (56 μL, 0.028 mmol, 0.02 equiv) were added and the mixture was stirred at 0° C. for 5 minutes. Cold 30% aqueous H₂O₂ (0.300 mL, 2.76 mmol, 2.00 equiv) was added dropwise then the ice bath was removed immediately (Noyori oxidation of 21 does not proceed at 0° C.) and the mixture was allowed to stir at 25° C. for 3 h. The reaction was checked for completion via TLC (50% ethyl acetate in hexanes; PAA stain) then brine (5 mL) was added. The mixture was stirred for 10 minutes then aqueous phase was discarded. The organic layer was dried over Na₂SO₄ then was concentrated via rotary evaporation to afford 22 as a light yellow oil (346 mg, 99%). ¹H NMR (CDCl₃, 300 MHz) δ 7.85 (dt, J=1.2, 7.2 Hz, 2H, SO₂Ph o-protons), 7.58 (t, J=7.2 Hz, 1H, SO₂Ph p-proton), 7.50 (t, J=6.9 Hz, 2H, SO₂Ph m-protons), 7.19 (t, J=6.3 Hz, 1H, C₆H, vinylsulfone proton), 6.04 (d, J=11.7 Hz, 1H, C₄H), 5.83 (dd, J=5.1, 11.7 Hz, 1H, C₃H), 3.81 (dt, J=3.3, 6.3 Hz, 1H, C₁H), 2.61 (dt, J=6.3, 16.5 Hz, 1H, C₇H), 2.51 (ddd, J=3.6, 5.1, 16.5 Hz, 1H, C₇H), 2.28 (hex., J=6.3 Hz, 1H, C₂H), 1.00 (d, J=6.9 Hz, 3H, C₈H, methyl protons), 0.92 (t, J=7.8 Hz, 9H, OTES methyl protons), 0.56 (q, J=8.1 Hz, 6H, OTES methylene protons); ¹³C NMR (CDCl₃, 75 MHz) δ 140.6, 140.2, 140.1, 138.5, 133.0, 129.0, 127.6, 118.7, 76.1, 43.6, 36.6, 18.9, 6.8, 4.8.

### Large Scale Procedure

[Amines and Halide Salts Interfere with this Reaction].

A 3-neck 2 L flask was fit with an overhead mechanical stirrer, and was placed in an ice-water bath. Dienyl sulfide 21 (65.0 g, 188.6 mmol, 1 equiv) was dissolved in toluene (600 mL), filtered onto a celite pad (This filtration is crucial since Noyori oxidation is halted by the presence of halide salts such as chlorides.) into the reaction flask, and the solution was cooled to 0° C. for 15 minutes. Aqueous 1M Na₂WO₄ (3.8 mL, 3.8 mmol, 0.020 equiv), aqueous 1M PhP(O)(OH)₂ (3.8 mL, 3.8 mmol, 0.02 equiv) and 0.5 M oct₃MeNHSO₄ in toluene (7.6 mL, 3.8 mmol, 0.02 equiv) were added and the mixture was stirred at 0° C. for 5 minutes. Cold 30% aqueous H₂O₂ (42.6 mL, 377.2 mmol, 2.00 equiv) was added dropwise then the reaction was allowed to warm up gradually over 2-3 hours without removing the ice bath (Removing the ice bath on large scale resulted in a sudden and vigorous reaction onset, leading to eruption.). The reaction was checked for completion via TLC (50% ethyl acetate in hexanes; PAA stain), stirred for additional 4 hours, then brine (5 mL) was added. The mixture was stirred for 10 minutes then aqueous phase was discarded. The organic layer was dried over Na₂SO₄ then was concentrated via rotary evaporation to afford 22 as a brown oil (71.7 g, 99%) that was sufficiently pure to carry to the next step. Silica pad filtration is highly recommended in order to remove a black baseline impurity, otherwise desilylation of crude product ensues. Silica pad filtration of the above crude oil (2.5×2.5 inch; 20% ethyl acetate in hexanes eluent) afforded 22 as a clear yellow oil of high purity as judged by ¹H NMR.

### Example 21: Synthesis of Epoxy Vinylsulfone 25-OTES/Ent-25-OTES

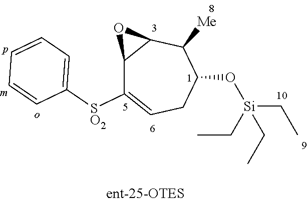 [SMILES: CC[Si](CC)(CC)O[C@@H]1CC=C(S(=O)(=O)c2ccccc2)[C@H]2O[C@H]2[C@H]1C]

A 3-neck 3 L flask was equipped with an overhead mechanical stirrer, a thermometer, and an argon inlet. Crude 22 (50.00 g, 132.3 mmol, 1 equiv) was dissolved in acid-free CH₂Cl₂ (330 mL) then was transferred to the flask, and cooled to 0° C. (internal temperature). After stirring for 10 minutes, (S,S)—Mn-salen catalyst (5.10 g, 7.94 mmol, 0.0600 equiv), 4-(3-phenylpropyl)pyridine N-oxide (8.90 g, 39.7 mmol, 0.300 equiv) were sequentially added. Cold 10% aqueous NaOCl (498.0 mL, 662.0 mmol, 5.00 equiv) was freshly mixed with cold 0.05 M aqueous NaH₂PO₄ (1195 mL), and the mixture was poured as a slow but continuous stream to the cold reaction giving a dark brown to black opaque solution. After 2 hours at 0° C., the reaction progress was checked via TLC (30% ethyl acetate in hexanes; PAA stain). It is noted that the reaction needs to be stopped until phase separation occurs, then the TLC sample must be withdrawn from the bottom organic layer via a Pasteur pipette. After completion, Hexanes (3× volume of DCM) was added to the reaction, and stirring was continued for 1-2 hours at 25° C. Ideally, Mn-salen catalyst precipitates as brown aggregates leaving a clear pale yellow solution above. If necessary, additional 10% aqueous NaOCl (0.5 equiv) is added at 25° C. in order to create a minor exotherm thereby destroying the residual Mn-salen catalyst, and preparing it for precipitation. Filtration over a celite pad (2.5×2.5 inch, hexane eluent) provides a clear yellow solution. Removal of solvents via rotary evaporation afforded ent-25-OTES as a clear yellow oil (43.3 g, 83%: estimated actual yield is 78% after next step; back calculation) that was reasonably pure for the next synthetic step. An analytical sample was obtained via flash column chromatography (30% ethyl acetate in hexanes) (Epoxide 23 partially decomposes on silica, thus must not be purified by flash chromatography.) as a clear oil. TLC (30% ethyl acetate in hexanes; PAA stain) R_{f} 0.4; ¹H NMR (CDCl₃, 300 MHz) δ 7.90 (dt, J=1.5, 7.2 Hz, 2H, SO₂Ph o-protons), 7.63 (tt, J=2.7, 9.9 Hz, 1H, SO₂Ph p-proton), 7.55 (tt, J=1.5, 6.9 Hz, 2H, SO₂Ph m-protons), 7.31 (ddd, J=0.6, 3.9, 8.4 Hz, 1H, C₆H, vinylsulfone proton), 3.69 (d, J=4.2 Hz, 1H, C₄H), 3.33 (dt, J=2.1, 9.0 Hz, 1H, C₁H), 3.26 (dd, J=3.3, 3.9 Hz, 1H, C₂H), 2.57 (ddd, J=2.1, 8.7, 17.1 Hz, 1H, C₇H), 2.43 (ddd, J=3.9, 9.0, 17.1 Hz, 1H, C₇H), 2.2 (m, 1H, C₂H), 1.15 (d, J=7.2 Hz, 3H, C₈H₃, methyl protons), 0.92 (t, J=7.8 Hz, 9H, OTES methyl protons), 0.57 (q, J=8.1 Hz, 6H, OTES methylene protons); ¹³C NMR (CDCl₃, 75 MHz) δ 141.2, 139.9, 139.4, 133.5, 129.2, 128.0, 70.8, 60.4, 52.2, 42.1, 38.1, 16.5, 6.7, 4.7.

### Example 22: Synthesis of Adduct 27/ent-27

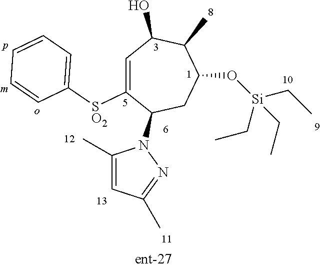 [SMILES: CC[Si](CC)(CC)O[C@@H]1C[C@@H](n2nc(C)cc2C)C(S(=O)(=O)c2ccccc2)=C[C@@H](O)[C@H]1C]

A 3-neck 1 L flask was fit with an overhead reflux condenser, and two septa. Crude ent-25-OTES (30.0 g, 76.1 mmol, 1 equiv) was dissolved in dry toluene (190 mL), then was transferred to the reaction flask. Dimethylpyrazole (3,5-DMP) (7.48 g, 66.1 mmol, 1 equiv) was added, then the reaction was heated at 50-60° C. for 1 hour and/or until TLC shows consumption of ent-25-OTES (Unnecessary heating after completion result in partial desilylation of 27.). The reaction was cooled to 25° C. then was washed with a mixture of crushed ice/brine (150 mL) and 5% aqueous HCl (45.0 mL, 60.9 mmol, 0.800 equiv). The aqueous phase was discarded, the organic phase was washed with brine, and dried over Na₂SO₄ for 3 hours. Removal of solvents via rotary evaporation afforded ent-27 as a crude brown oil. Purification via silica pad filtration (2.5×2.5 inch, 30% ethyl acetate in hexanes) afforded ent-27 as a highly pure crystalline solid (24.9 g, 77%) (see scheme 8 above) that was carried to the next synthetic step. [α]²⁵ +89.8 (DCM, c=1), ¹H NMR (CDCl₃, 300 MHz) δ 7.74 (d, J=8.1 Hz, 1H, C₄H, vinylsulfone proton), 7.52 (d, J=8.4 Hz, 2H, SO₂Ph o-protons), 7.40 (t, J=7.8 Hz, 1H, SO₂Ph p-proton), 7.27 (t, J=7.5 Hz, 2H, SO₂Ph m-protons), 6.92 (d, J=9.9 Hz, 1H, hydroxyl proton), 5.44 (s, 1H, C₁₃H), 5.31 (t, J=4.2 Hz, 1H, C₆H), 4.34 (t, J=7.2 Hz, 1H, C₃H), 3.94 (dt, J=2.4, 9.9 Hz, 1H, C₁H), 2.15 (m, 1H, C₇H), 2.10 (s, 3H, C₁₂H₃, methyl protons), 2.05 (m, 1H, C₇H), 1.92 (s, 3H, C₁₁H₃, methyl protons), 1.80 (p, J=9.0 Hz, 1H, C₂H), 1.20 (d, J=6.6 Hz, 3H, C₈H₃, methyl protons), 0.77 (t, J=7.5 Hz, 9H, OTES methyl protons), 0.36 (q, J=7.5 Hz, 6H, OTES methylene protons); ¹³C NMR (CDCl₃, 75 MHz) δ 147.0, 146.3, 140.1, 138.8, 138.6, 132.9, 128.5, 127.0, 105.3, 68.7, 67.3, 50.5, 45.8, 44.2, 16.5, 12.8, 10.5, 6.6, 4.6.

### Example 23: Synthesis of Stereotetrad 28a/Ent-28a

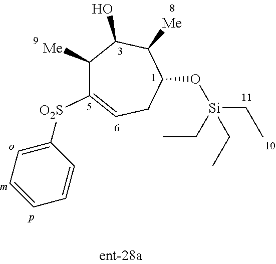 [SMILES: CC[Si](CC)(CC)O[C@@H]1CC=C(S(=O)(=O)c2ccccc2)[C@@H](C)[C@@H](O)[C@H]1C]

This Procedure is Sensitive to the Degree of Purity of Ent-27.

A 3-neck 100 mL round bottom was fit with a reflux condenser and two rubber septa, then was flame dried under a stream of dry N₂. After cooling to 25° C., a solution of ent-27 (8.0 g, 16.4 mmol, 1 equiv) in dry toluene (177 mL) was transferred to the flask via cannula. The solution was brought to a temperature of 50° C. (internal temperature) then 1.4 M MeMgBr in toluene/THF (25.9 mL, 36.1 mmol, 2.20 equiv) was added at a rate of 2 mL/min (Grignard addition was effected via a liquid addition pump.) such that the internal temperature did not exceed 50° C. The reaction was checked for progress once MeMgBr addition was complete and was found complete.

Quenching at 50° C. with saturated aqueous NH₄Cl (18 mL, careful dropwise addition) was followed by addition of excess saturated aqueous NH₄Cl (300 mL) and/or until all magnesium salts dissolved. The aqueous layer was back extracted with ether (2×100 mL) then was discarded. The combined organic layers were washed with aqueous 5% HCl, brine then dried over Na₂SO₄ for 4 hours. Removal of solvents via rotary evaporation afforded ent-28a as a yellow oil (6.3 g, 94%) that was judged highly pure by ¹H NMR. An analytical sample was obtained via flash column chromatography (30% ethyl acetate in hexanes) as a clear oil. TLC (40% ethyl acetate in hexanes) R_{f} 0.4; ¹H NMR (CDCl₃, 300 MHz) δ 7.84 (dt, J=1.5, 7.2 Hz, 2H, SO₂Ph o-protons), 7.58 (tt, J=0.9, 7.5 Hz, 1H, SO₂Ph p-proton), 7.49 (tt, J=1.2, 8.4 Hz, 2H, SO₂Ph m-protons), 7.12 (dd, J=6.0, 8.1 Hz, 1H, C₆H, vinylsulfone proton), 3.81 (dt, J=1.8, 6.9 Hz, 1H, C₃H), 3.75 (t, J=3.9 Hz, 1H, C₁H), 2.79 (dq, J=3.6, 7.5 Hz, 1H, C₄H), 2.70 (ddd, J=1.8, 6.0, 15.3 Hz, 1H, C₇H), 2.52 (m, 1H, C₇H), 2.00 (m, 1H, C₂H), 1.82 (s, 1H, hydroxyl proton), 1.19 (d, J=7.5 Hz, 3H, C₉H₃, methyl protons), 1.06 (d, J=7.5 Hz, 3H, C₈H₃, methyl protons), 0.94 (t, J=8.1 Hz, 9H, OTES methyl protons), 0.58 (q, J=8.1 Hz, 6H, OTES methylene protons); ¹³C NMR (CDCl₃, 75 MHz) δ 143.8, 139.9, 139.5, 133.0, 129.0, 127.7, 71.9, 69.9, 47.3, 40.1, 31.8, 15.0, 14.4, 6.8, 4.7.

### Example 24: Synthesis of Stereotetrad 2/Ent-2

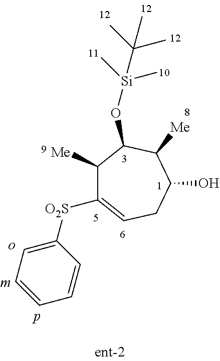 [SMILES: C[C@@H]1[C@H](O[Si](C)(C)C(C)(C)C)[C@H](C)C(S(=O)(=O)c2ccccc2)=CC[C@H]1O]

A dry 100 mL round bottom flask was charged with a solution of 28a (6.30 g, 15.4 mmol, 1 equiv) in dry DCM (60 ml), then the solution was cooled to 0° C. After 15 minutes, 2,6-lutidine (2.30 mL, 19.1 mmol, 1.24 equiv) was added dropwise, then TBSOTf (3.60 mL, 15.4 mmol, 1 equiv) was added dropwise via syringe. The reaction was stirred at 0° C. for 30-60 minutes, then MeOH (˜3.20 mL, 77.0 mmol, 5.00 equiv) was added dropwise, and stirring was continued for another 30 minutes. The reaction was diluted with 5% aqueous HCl, washed, then the aqueous layer was discarded after neutralization. The organic layer was transferred to another flask, cooled to 0° C., then CSA (1.76 g, 7.70 mmol, 0.500 equiv) was added.

After stirring at 0° C. for 2 hours, the reaction was neutralized carefully with sat aqueous NaHCO₃, then washed with saturated aqueous NaHCO₃. The organic layer was then washed with brine, then dried over Na₂SO₄ for 6 hours. Removal of solvents via rotary evaporation afforded crude 2 as a brown oil. Purification by flash column chromatography afforded 2 as a clear viscous oil (4.78 g, 76% over 2 steps); [α]²⁵ −80.1 (DCM, c=1), ¹H NMR (CDCl₃, 300 MHz) δ 7.87 (dt, J=1.2, 6.9 Hz, 2H, SO₂Ph o-protons), 7.61 (tt, J=1.2, 7.5 Hz, 1H, SO₂Ph p-proton), 7.53 (tt, J=1.8, 7.2 Hz, 2H, SO₂Ph m-protons), 7.15 (t, J=6.3 Hz, 1H, C₆H, vinylsulfone proton), 3.90 (m, 1H, C₁H), 3.77 (dd, J=4.2, 6.0 Hz, 1H, C₃H), 2.76 (ddd, J=2.4, 5.7, 16.2 Hz, 1H, C₄H), 2.63 (dt, J=6.9, 17.1 Hz, 1H, C₇H), 2.54 (ddd, J=3.6, 7.8, 14.7 Hz, 1H, C₇H), 2.07 (hex, J=6.9 Hz, 1H, C₂H), 1.72 (d, J=4.2 Hz, 1H, hydroxyl proton), 1.21 (d, J=6.9 Hz, 3H, C₉H₃, methyl protons), 1.13 (d, J=7.5 Hz, 3H, C₈H₃, methyl protons), 0.80 (s, 9H, OTBS tert-butyl protons), −0.19 (s, 3H, OTBS methyl protons), −0.35 (s, 3H, OTBS methyl protons); ¹³C NMR (CDCl₃, 75 MHz) δ 145.2, 139.3, 138.2, 133.2, 129.2, 128.0, 72.4, 68.9, 47.4, 41.4, 32.1, 25.6, 17.9, 15.2, 13.5, −5.3, −5.5.

### Example 25: Synthesis of Diol 4/Ent-4 from Vinylsulfone 28a/Ent-28a

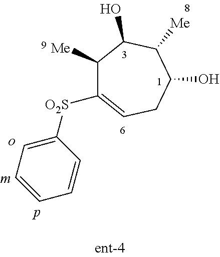 [SMILES: C[C@H]1[C@H](O)[C@H](C)C(S(=O)(=O)c2ccccc2)=CC[C@H]1O]

A 15 mL round bottom flask was charged with a solution of ent-28a (300 mg, 0.800 mmol, 1 equiv) in CH₂Cl₂ (8 mL) at 25° C. The solution was cooled to 0° C. then CSA (189.6 mg, 0.800 mmol, 1 equiv) was added as a solid or solution in CH₂Cl₂. After five minutes, MeOH (5 drops) was added and stirring was continued at 0 to 25° C. for 3 hours. After checking completion by TLC (50% ethyl acetate in hexanes; PAA stain), saturated aqueous NaHCO₃ was added. The biphasic mixture was transferred to a separatory funnel, shaken then the aqueous phase was discarded. The organic layer was washed with brine, dried over Na₂SO₄ then concentrated via rotary evaporation. The resulting crude oil was purified via flash column chromatography (50% ethyl acetate in hexanes) and afforded the above diol ent-4 as a clear yellow oil (199.1 mg, 92%). TLC (50% ethyl acetate in hexanes; PAA stain) R_{f} 0.12; ¹H NMR (CDCl₃, 300 MHz) δ 7.87 (dt, J=1.5, 7.2 Hz, 2H, SO₂Ph o-protons), 7.62 (tt, J=1.5, 7.2 Hz, 1H, SO₂Ph p-proton), 7.54 (tt, J=1.5, 6.9 Hz, 2H, SO₂Ph m-protons), 7.15 (t, J=6.9 Hz, 1H, C₆H, vinylsulfone proton), 3.82 (t, J=3.3 Hz, 1H, C₃H), 3.76 (dt, J=2.4, 7.5 Hz, 1H, C₁H), 2.94 (dq, J=3.3, 7.5 Hz, 1H, C₄H), 2.78 (ddd, J=2.4, 6.9, 15.0 Hz, 1H, C₇H), 2.59 (dt, J=7.5, 15.0 Hz, 1H, C₇H), 1.97 (bs, 2H, hydroxyl protons), 1.91 (dp, J=3.6, 6.9 Hz, 1H, C₂H), 1.16 (d, J=2.1 Hz, 3H, C₉H₃, methyl protons), 1.13 (d, J=2.4 Hz, 3H, C₈H₃, methyl protons); ¹³C NMR (CDCl₃, 75 MHz) δ 145.1, 139.6, 138.9, 133.3, 129.2, 127.9, 72.1, 70.8, 46.6, 39.9, 33.3, 15.5, 15.3; LRMS (CI) calculated for C₁₅H₂₀O₄S, 296. found [M-H₂O]+ 278; HRMS (CI) calculated for [C₁₅H₂₀O₄Si—H₂O]+ 278.0977. found 278.0984.

### Example 26: Synthesis of 33

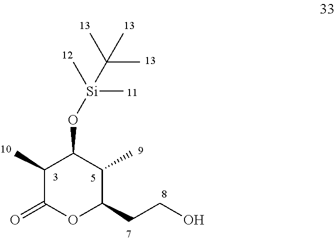 [SMILES: C[C@H]1[C@H](O[Si](C)(C)C(C)(C)C)[C@H](C)C(=O)O[C@@H]1CCO]

### Ozonolysis in Acetone

3-neck 1 L flask was fit with an overhead thermometer, an ozone inlet, and an ozone outlet that was attached to a jar containing Na₂SO₃ aqueous solution. The flask was charged with 16 (5.00 g, 12.2 mmol, 1 equiv) then with acetone (244 mL)(Acetone must be distilled from anhydrous CaSO₄.), and NaHCO₃ (2.00 g, 24.4 mmol, 2.00 equiv). The flask was placed in a dry ice-acetone bath while the internal temperature was kept between −30 to −35° C. At this stage, ozone was bubbled until a faint blue solution persists then the consumption of 9 was monitored by TLC (30-50% ethyl acetate in hexanes; PAA stain). Ozone bubbling was stopped, and the reaction was purged with dry N₂ for 30-45 minutes at −30° C. then dimethyl sulfide Me₂S (803 μL, 11.0 mmol, 0.900 equiv) was added. The ice bath was removed, and the reaction was allowed to stir at 25° C. under Ar for 12 hours. After checking TLC for completion, the reaction was concentrated via rotary evaporation until all acetone was removed. Vacuum was broken under Ar, then the residue was dissolved in DCM (60 mL), and the reaction was cooled to 0° C. After 15 minutes, ^{t}BuNH₂.BH₃ (16.5 g, 18.3 mmol, 0.75 equiv) was added, and the reaction was stirred at 0 to 25° C. for 60 minutes.

After completion, aqueous 5% HCl was added dropwise with caution, then the organic phase was washed with aqueous 5% HCl, brine, then dried over Na₂SO₄ for 2 hours. Removal of solvents via rotary evaporation afforded crude 33 as a highly pure clear oil. An analytical sample was obtained via flash column chromatography (50% ethyl acetate in hexanes) to give 33 as a clear colorless oil (80% recovery)(In order to obtain maximum yields, precautions must be considered. The ozone generator must be started 5-10 minutes before passing ozone into the reaction flask in order to avoid aldehyde oxidation by molecular oxygen. Ozonolysis must be stopped once starting material is consumed, otherwise lower yields ensue. Nitrogen purging for 45 minutes is essential to get rid of all ozone before introduction of Me₂S.). TLC (50% ethyl acetate in hexanes; PAA stain) R_{f} 0.32; ¹H NMR (CDCl₃, 300 MHz) δ 4.01 (dt, J=2.4, 9.6 Hz, 1H, C₆H), 3.90 (dt, J=4.5, 10.8 Hz, 1H, C₈H), 3.85 (dt, J=5.1, 10.8 Hz, 1H, C₈H), 3.68 (dd, J=2.1, 2.4 Hz, 1H, C₄H), 2.63 (dq, J=3.3, 6.9 Hz, 1H, C₃H), 1.98 (dddd, J=2.4, 5.4, 8.1, 14.1 Hz, 1H, C₅H), 1.85 (dt, J=4.8, 9.3 Hz, 1H, C₇H), 1.80 (dt, J=4.5, 9.3 Hz, 1H, C₇H), 1.22 (d, J=6.6 Hz, 3H, C₁₀H₃, methyl protons), 1.03 (d, J=7.2 Hz, 3H, C₉H₃, methyl protons), 0.87 (s, 9H, OTBS tert-butyl protons), 0.07 (s, 3H, OTBS methyl protons), 0.05 (s, 3H, OTBS methyl protons); ¹³C NMR (CDCl₃, 75 MHz) δ 174.4, 79.0, 76.5, 58.7, 42.5, 39.1, 36.4, 25.6, 17.8, 15.7, 12.1, −4.4, −4.9.

### Ozonolysis in Dichloromethane

Ozone was bubbled into a solution of 16 (5.50 g, 13.6 mmol, 1 equiv) in dichloromethane (220 mL) at −40° C. for 60 minutes. Nitrogen was then bubbled for 30 minutes to purge ozone from the solution, followed by addition dimethyl sulfide (9.96 mL, 136 mmol, 10.0 equiv) and the mixture stirred at room temperature under for 8 hours. After completion, ^{t}BuNH₂.BH₃ (2.35 g, 27.2 mmol, 2.00 equiv) was added next and the reaction stirred for 60 more minutes. The reaction mixture was quenched with aqueous 5% HCl and extracted with dichloromethane (2×100 mL). The organic extracts were dried with a mixture of Na₂SO₄ and K₂CO₃ and concentrated using a rotary evaporator to give a light yellow oil. The crude oil was purified by flash column chromatography (hexanes:ethyl acetate 1:1→2:3) to afford 2.86 g (70%) of 33.

### Example 27: Synthesis of 34/Ent-34

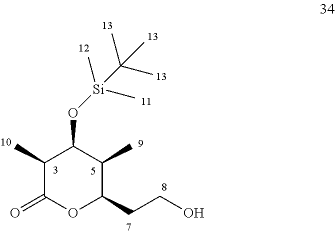 [SMILES: C[C@@H]1[C@H](O[Si](C)(C)C(C)(C)C)[C@H](C)C(=O)O[C@@H]1CCO]

### Ozonolysis in Acetone

A 3-neck 1 L flask was fit with an overhead thermometer, an ozone inlet, and an ozone outlet that was attached to a jar containing Na₂SO₃ aqueous solution. The flask was charged with 2/ent-2 (5.00 g, 12.2 mmol, 1 equiv) then with acetone (240 mL) (acetone must be distilled from anhydrous CaSO₄), and NaHCO₃ (1.00 g, 24.4 mmol, 2.00 equiv). The flask was placed in a dry ice-acetone bath while the internal temperature was kept at −30 to −35° C. At this stage, ozone was bubbled until a persistent blue (faint blue) solution persists then the consumption of 2/ent-2 was monitored by TLC (30-50% ethyl acetate in hexanes; PAA stain). Ozone bubbling was stopped, and the reaction was purged with dry N₂ for 45 minutes at −30° C. then dimethylsulfide (4.50 mL, 61.0 mmol, 5.00 equiv). The ice bath was removed, and the reaction was allowed to stir at 25° C. under Ar for 12 hours.

After checking TLC for completion, the reaction was concentrated via rotary evaporation until all acetone was removed. Vacuum was broken under Ar, then the residue was dissolved in DCM (60 mL), and the reaction was cooled to 0° C. After 15 minutes, ^{t}BuNH₂.BH₃ (1.60 g, 18.3 mmol, 1.50 equiv) was added, and the reaction was stirred at 0 to 25° C. for 60 minutes. After completion, aqueous 5% HCl was added dropwise with caution, then the organic phase was washed with aqueous 5% HCl, brine, then dried over Na₂SO₄ for 2 hours. Removal of solvents via rotary evaporation afforded crude 34 as a highly pure clear oil. An analytical sample was obtained via flash column chromatography (50% ethyl acetate in hexanes) to give 34 as a clear colorless oil (76% recovery). TLC (50% ethyl acetate in hexanes; PAA) R_{f} 0.28.

### Ozonolysis in Ethylacetate

Ozone was bubbled into a solution of 2 (3.3 g, 8.0 mmol, 1 equiv) in ethylacetate (85 mL) at −40° C. for 30 minutes. Nitrogen was then bubbled for 15 minutes to purge ozone from the solution, followed by addition dimethyl sulfide (2.95 mL, 40.2 mmol, 5.00 equiv) and the mixture stirred at room temperature. After 8 hours, t-BuNH₂.BH₃ (1.05 g, 12.1 mmol, 1.50 equiv) was added next and the reaction was stirred for 30 more minutes. The reaction mixture was quenched with aqueous 5% HCl and extracted with ethylacetate (2×60 mL). The organic extracts were dried with a mixture of Na₂SO₄ and K₂CO₃ and concentrated using a rotary evaporator to give a light yellow oil. The crude oil was purified by flash column chromatography (hexanes: ethyl acetate 1:1→2:3) to afford 1.59 g (65%) of 34.

It will be appreciated by those skilled in the art that changes could be made to the embodiments described above without departing from the broad inventive concept thereof. It is understood, therefore, that this invention is not limited to the particular embodiments disclosed, but it is intended to cover modifications that are within the spirit and scope of the invention, as defined by the appended claims.

What is claimed is:

1. A compound of formula (XI) 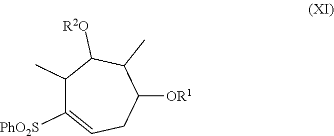
wherein R¹ and R² are independently H or a hydroxyl protecting group, wherein R¹ and R² can be same or different hydroxyl protecting group.

2. The compound of claim 1, wherein the compound has a formula (XII)

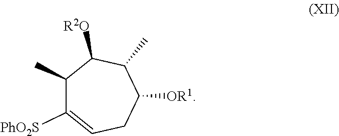

3. The compound of claim 1, wherein the compound has a formula (XIII)

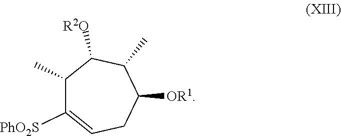

4. The compound of claim 1, wherein the compound has a formula (XIV)

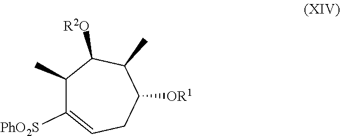

5. The compound of claim 1, wherein the hydroxyl protecting group and the hydroxyl group being protected forms a C—O ether bond, a Si—O silyl ether bond, or —(C═O)—O acyl bond, or any combination thereof.

6. The compound of claim 1, wherein the protecting group is selected from the group consisting of trimethylsilyl (TMS), triethylsilyl (TES), tert-butyl-dimethylsilyl (TBS), triisopropylsilyl (TIPS), tert-butyl-diphenylsilyl (TBDPS), triphenylsilyl, dimethylphenylsilyl, methyldiphenylsilyl, acetyl (Ac), pivaloyl (piv), trichloroacetyl, 2,2,2-trichloroethoxycarbonyl (Troc), benzyl, p-methoxybenzyl (PMB), 3-phenylsulfonylpropionyl, benzoyl (Bz), benzyl (Bn), beta-methoxyethoxyl (MEM), dimethoxytrityl (DMT), methoxymethyl (MOM), p-methoxybenzyl (PMB), tetrahydropyranyl (THP), tetrahydrofuranyl (THF), ethoxyethyl (EE), and any combination thereof.

7. A composition comprising the compound of claim 1.
